(19) United States
(12) Patent Application Publication
(10) Pub. No.: US 2023/0101833 A1
(43) Pub. Date: Mar. 30, 2023

(54) CHEMICAL TREATMENT FOR PREPARING METAL ELECTRODES

(71) Applicant: Commonwealth Scientific and Industrial Research Organisation, Acton (AU)

(72) Inventors: Adam Best, Acton (AU); Gavin Collis, Acton (AU)

(21) Appl. No.: 17/797,057

(22) PCT Filed: Feb. 3, 2021
(86) PCT No.: PCT/AU2021/050083

§ 371 Date: Aug. 2, 2022

(30) Foreign Application Priority Data
Feb. 3, 2020 (AU) 2020900286

(51) Int. Cl. C01D 3/22; C01D 3/02; H01M 10/052

(52) U.S. Cl. CPC C01D 3/22; C01D 3/02; H01M 10/052; C01P 2006/40

(57) ABSTRACT

The present disclosure relates to chemical treatments for preparing metal electrodes, including ex-situ chemical treatments for preparing metal electrodes and to ex-situ chemically treated metal electrodes, which can be used in electrochemical cells. The present disclosure also relates to methods for forming a metal fluoride-based layer (e.g. a SEI fluoride layer) on a metal or an electrode thereof comprising an ex-situ chemical treatment of the metal or electrode thereof, and to electrochemical cells comprising the ex-situ chemically treated metal electrodes.

### FIELD

The present disclosure relates to chemical treatments for preparing metal electrodes, and in particular ex-situ chemical treatments for preparing metal electrodes and to ex-situ chemically treated metal electrodes, which can be used in electrochemical cells. The present disclosure also relates to a method for forming a metal fluoride-based layer (or Solid Electrolyte Interphase (SET)) on a metal or an electrode thereof comprising an ex-situ chemical treatment of the metal or electrode thereof. The present disclosure also relates to electrochemical cells comprising the ex-situ chemically treated metal electrodes.

### BACKGROUND

The performance of many electrochemical cells, such as primary and secondary batteries, is often dictated by chemical reactions between the metal electrodes and electrolyte. In particular, the electrolyte can react with the surface of the metal electrode resulting in the in-situ formation of a solid electrolyte interphase (SEI) on the metal electrode surface, the composition of which is highly dependent on the electrolytes and additives present in the electrochemical cell. The SEI layer is typically comprised of inorganic and organic constituents, some of which are more robust than others. Depending on the electrolytes and additives that generate the SEI layer in-situ, during cycling there may be a steady consumption of metal an electrolyte which leads to a loss of capacity and low Coulombic efficiency (CE).

Additionally, during cycling, the metal electrodepositions/dissolution process may not be uniform on various metal electrodes, leading to the formation of high surface area metal on the electrode resulting in “dead metal”, which no longer participates in the deposition and dissolution process, and in some cases results in the formation of metal dendrites which can grow through the separator leading to an internal short-circuit by reaching the cathode, which can result in thermal runaway, causing serious safety problems. For other metal electrodes, the SEI layer may prevent the recharging process.

There is a need for alternative or improved processes to access SEI modified electrodes for use in electrochemical cells, and methods for preparing SEI modified electrodes for electrochemical cells, which are scalable for industrial application and flexible for providing control over properties and performance.

It will be understood that any prior art publications referred to herein do not constitute an admission that any of these documents form part of the common general knowledge in the art, in Australia or in any other country.

### SUMMARY

The present inventors have undertaken research and development into methods for preparing improved solid-electrolyte interphases (SEI) on the surface of various metals. The metals can be used as metal electrodes in electrochemical cells, for example in in primary and/or secondary (rechargeable) batteries. The presence of the SEI layer on the metal electrode surface can lead to improved cycling and performance when used in an electrochemical cell, for example in primary and/or secondary (rechargeable) batteries. In particular, the present inventors have identified that ex-situ treatment of the surface of various metals or electrodes thereof with one or more fluorinating agents can produce an improved surface SEI layer. The methods as described herein can be scalable for industrial application and can provide for control, flexibility and consistency in the manufacture of ex-situ chemically treated metal or electrodes thereof.

In one aspect, there is provided a method for forming an ex-situ SEI fluoride layer on the surface of a metal or an electrode thereof, the method comprising the step of contacting the surface of the metal with an organic solvent preparation comprising one or more fluorinating agents.

In an embodiment, the metal is a metal electrode. In another embodiment, the metal is a metal sheet. It will be appreciated that the metal sheet is suitable for use in preparing a metal electrode.

In an embodiment, the metal is a metal is selected from the group consisting of metals of Group 1, Group 2, or Group 13 of the Periodic Table of Elements. In an embodiment, the metal is an alkali metal or an alkali earth metal. In another embodiment, the metal is selected from the group consisting of lithium, magnesium, calcium, sodium, aluminium, and potassium metal. In an embodiment, the metal is lithium metal or magnesium metal. In an embodiment, the metal is lithium metal.

In an embodiment, the organic solvent preparation comprises one or more aprotic organic solvents. The one or more aprotic solvents may comprise or consist of one or more ionic liquids.

In another aspect, there is provided a method for forming an ex-situ SEI fluoride layer on the surface of a metal or an electrode thereof, the method comprising the step of contacting the surface of the metal with an organic solvent preparation consisting of one or more aprotic organic solvents, one or more fluorinating agents, and optionally one or more additives.

In another aspect, there is provided an ex-situ SEI fluoride layered metal or an electrode thereof. The ex-situ SEI fluoride layered metal or an electrode thereof may be prepared according to any embodiments or examples of the methods and device as described herein. For example, the ex-situ SEI fluoride layered metal electrode can comprise or consist of an ex-situ SEI fluoride layer on a surface of the metal. The metal may be configured as an electrode.

In another aspect, there is provided a method of assembling an electrochemical cell comprising a metal electrode, whereby the steps comprise:

treating metal or an electrode thereof according to any of the embodiments or examples thereof as described herein to form an ex-situ SEI fluoride layered metal or electrode thereof;

optionally preparing an ex-situ SEI fluoride layered metal electrode from the ex-situ SEI fluoride layered metal; and

assembling the ex-situ SEI fluoride layered metal electrode into an electrochemical cell.

In another aspect, there is provided an electrochemical cell comprising:

a negative electrode that is an ex-situ SEI fluoride layered metal electrode according to any embodiments or examples as described herein;

a positive electrode comprising a positive electrode active material; and

an electrolyte comprising one or more electrolyte solvents.

It will be appreciated that other aspects, embodiments and examples of the methods, treatments, electrodes and devices are described herein.

It will be appreciated that embodiments as described herein in relation to method for forming an ex-situ SEI fluoride layer on the surface of a metal or an electrode thereof, including embodiments relating to the metal, fluorinating agent, SEI metal fluoride layer, and various process steps, can also equally provide embodiments for the ex-situ SEI fluoride layered metal or an electrode thereof, the method of assembling an electrochemical cell comprising a metal electrode, and/or the electrochemical cell, as defined below, and vice versa.

### BRIEF DESCRIPTION OF THE DRAWINGS

Preferred embodiments of the present disclosure are further described and illustrated as follows, by way of example only, with reference to the accompanying drawings in which:

FIG. 1: Overvoltage of a symmetrical Li∥Li cell with non-ex-situ modified lithium metal electrodes in an IL-based electrolyte.

FIG. 2: Evolution of interfacial resistance during cycling of symmetrical Li∥Li cells with an IL-based electrolyte (Pyr₁₃FSI) with and without additives (LiNO₃) on the lithium metal electrodes (prior art comparison).

FIG. 3: Overvoltage of selected cycles of symmetrical Li∥Li cells with Pyr₁₃FSI-based lithium electrolytes with and without additives (prior art comparison).

FIG. 4: Overvoltage at different C-rates in an IL-based electrolyte for lithium metal electrodes a) without fluorinating agents (only Pyr₁₃FSI-based chemicals) and b) with Fluorinating agent, TBAF, and different solvent/salt combinations.

FIG. 5: Evolution of interfacial resistance of symmetrical Li∥Li cells in an IL-based electrolyte with two different fluorinating agents; a) FPT based modifications and b) TBAF-based modifications on the lithium metal electrodes during cycling.

FIG. 6: Overvoltage of selected cycles of symmetrical Li∥Li cells with different FPT or/and TBAF-based lithium modifications in IL electrolytes/solvents.

FIG. 7: Overvoltage of selected cycles of symmetrical Li∥Li cells with different TBAF-based lithium modifications in an IL-based electrolyte with different solvents.

FIG. 8: Overvoltage of selected cycles of symmetrical Li∥Li cells with FPT-based lithium modifications in an IL-based electrolyte with different solvents.

FIG. 9: Specific discharge capacity of Li∥S cells in an organic electrolyte with lithium metal electrodes a) modified with IL only and b) modified with fluorinating agents.

FIG. 10: Voltage plateaus of selected cycles in an organic electrolyte in Li∥S cells with lithium metal anode modified a) IL-based, b) FPT-based and c) TBAF-based.

FIGS. 11 to 13: Survey XPS spectra of Li electrode surfaces following precleaning with various solvents, prior to immersion with fluorinating agent.

FIG. 14. High-resolution Lithium, Fluorine, Carbon and Nitrogen XPS spectra of pre-cleaned Li electrode surface following immersion with fluorinating agent.

FIGS. 15 and 16. XPS spectra and analysis of Mg surface highlighting effect fluorinating agents and non-fluorinating agents have on SEI layer composition.

FIG. 17. Survey XPS spectra of Li electrode surfaces highlighting effect fluorinating agents and non-fluorinating agents have on SEI layer composition.

FIG. 18: Survey XP spectra of Li electrode surface exposed to TBAF (fluorinating agent) either alone or with an additive (TBANO₃).

### DETAILED DESCRIPTION

The present disclosure describes the following various non-limiting embodiments, which relate to investigations undertaken to identify improvements in the performance of metal electrodes, which can be used in electrochemical cells, particularly secondary (rechargeable) batteries. It was surprisingly found that a scalable, flexible and effective industrial method could be provided for pre-treating metal or an electrode thereof, prior to use of the metal as electrodes in an electrochemical cell. The ex-situ treated metal electrodes comprise an effective solid electrolyte interphase (SEI) layer on the surface of the electrodes, which can subsequently be assembled into an electrochemical cell. The methods as described herein provide an ex-situ formation of a metal fluoride containing SEI layer on the surface of the metal or an electrode thereof. The methods comprise an organic solvent preparation comprising one or more fluorinating agents, which is further described below according to various non-limiting embodiments and examples. At least according to some embodiments or examples as described herein, the methods provide for a relatively controlled, safe and low temperature industrial scale preparation of SEI fluoride layered lithium metal or electrodes thereof.

### Terms

In the following description, reference is made to the accompanying drawings which form a part hereof, and which is shown, by way of illustration, several embodiments. It is understood that other embodiments may be utilized and structural changes may be made without departing from the scope of the present disclosure.

With regards to the definitions provided herein, unless stated otherwise, or implicit from context, the defined terms and phrases include the provided meanings. Unless explicitly stated otherwise, or apparent from context, the terms and phrases below do not exclude the meaning that the term or phrase has acquired by a person skilled in the relevant art. The definitions are provided to aid in describing particular embodiments, and are not intended to limit the claimed invention, because the scope of the invention is limited only by the claims. Furthermore, unless otherwise required by context, singular terms shall include pluralities and plural terms shall include the singular.

All publications discussed and/or referenced herein are incorporated herein in their entirety.

Any discussion of documents, acts, materials, devices, articles or the like which has been included in the present specification is solely for the purpose of providing a context for the present disclosure. It is not to be taken as an admission that any or all of these matters form part of the prior art base or were common general knowledge in the field relevant to the present disclosure as it existed before the priority date of each claim of this application.

Throughout this disclosure, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the disclosure herein is susceptible to variations and modifications other than those specifically described. It is to be understood that the disclosure includes all such variations and modifications. The disclosure also includes all of the examples, steps, features, methods, compositions, coatings, processes, and coated substrates, referred to or indicated in this specification, individually or collectively, and any and all combinations or any two or more of said steps or features.

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, typically refers to +/−10%, for example +/−5%, of the designated value.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout the present specification, various aspects and components of the invention can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 5, 5.5 and 6, unless where integers are required or implicit from context. This applies regardless of the breadth of the disclosed range. Where specific values are required, these will be indicated in the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “alkyl” includes straight-chained, branched, and cyclic alkyl groups and includes both unsubstituted and substituted alkyl groups. Unless otherwise indicated, the alkyl groups typically contain from 1 to 20 carbon atoms. The alkyl groups may for example contain carbon atoms from 1 to 12, 1 to 10, or 1 to 8. Examples of “alkyl” as used herein include, but are not limited to, methyl, ethyl, n-propyl. n-butyl, n-pentyl, isobutyl, t-butyl, isopropyl, n-octyl, n-heptyl, ethylhexyl, cyclopentyl, cyclohexyl, cyclo heptyl, adamantyl, and norbornyl, and the like. Unless otherwise noted, alkyl groups may be mono- or polyvalent.

As used herein, the terms “halo” or “halogen”, whether employed alone or in compound words such as haloalkyl, means fluorine, chlorine, bromine or iodine.

As used herein, the term “haloalkyl” means an alkyl group having at least one halogen substituent, the terms “alkyl” and “halogen” being understood to have the meanings outlined above. Similarly, the term “monohaloalkyl” means an alkyl group having a single halogen substituent, the term “dihaloalkyl” means an alkyl group having two halogen substituents and the term “trihaloalkyl” means an alkyl group having three halogen substituents. Examples of monohaloalkyl groups include fluoromethyl, chloromethyl, bromomethyl, fluoromethyl, fluoropropyl and fluorobutyl groups; examples of dihaloalkyl groups include difluoromethyl and difluoroethyl groups; examples of trihaloalkyl groups include trifluoromethyl and trifluoroethyl groups.

As used herein, the terms “carbocyclic” and “carbocyclyl” represent a ring system wherein the ring atoms are all carbon atoms, e.g., from 3 to 20 carbon ring atoms, and which may be aromatic, non-aromatic, saturated, or unsaturated. The terms encompass single ring systems, e.g. cycloalkyl groups such as cyclopentyl and cyclohexyl, aromatic groups such as phenyl, and cycloalkenyl groups such as cyclohexenyl, as well as fused-ring systems such as naphthyl and fluorenyl.

As used herein, the terms “heterocyclic” and “heterocyclyl” represent an aromatic or a non-aromatic cyclic group of carbon atoms wherein from one to three of the carbon atoms is/are replaced by one or more heteroatoms independently selected from nitrogen, oxygen or sulfur. A heterocyclyl group may, for example, be monocyclic or polycyclic, and contain for example from 3 to 20 ring atoms. In a bicyclic heterocyclyl group there may be one or more heteroatoms in each ring, or only in one of the rings. Examples of heterocyclyl groups include piperidinyl, tetrahydrofuranyl, tetrahydropyranyl, pyridyl, pyrimidinyl and indolyl.

As used herein, the term “cycloalkyl” represents a ring system wherein the ring atoms are all carbon atoms, e.g., from 3 to 20 carbon ring atoms, and which is saturated. A cycloalkyl group can be monocyclic or polycyclic. A bicyclic group may, for example, be fused or bridged. Examples of monocyclic cycloalkyl groups include cyclopropyl, cyclobutyl and cyclopentyl. Other examples of monocyclic cycloalkyl groups are cyclohexyl, cycloheptyl and cyclooctyl. Examples of bicyclic cycloalkyl groups include bicyclo[2.2.1]hept-2-yl.

As will be understood, an “aromatic” group means a cyclic group having 4n+2π electrons, where n is an integer equal to or greater than 1. As used herein, “aromatic” is used interchangeably with “aryl” to refer to an aromatic group, regardless of the valency of aromatic group.

As used herein, the terms “aromatic carbocyclyl” or “aromatic carbocycle” represent a ring system which is aromatic and in which the ring atoms are all carbon atoms, e.g. having from 6-14 ring atoms. An aromatic carbocyclyl group may be monocyclic, bicyclic or polycyclic. Examples of aromatic carbocyclyl groups include phenyl, naphthyl and fluorenyl. Polycyclic aromatic carbocyclyl groups include those in which only one of the rings is aromatic, such as for example indanyl.

The term “aryl” or “aromatic” group or moiety includes 6-18 ring atoms and can contain optional fused rings, which may be saturated or unsaturated. Examples of aromatic groups include phenyl, naphthyl, biphenyl, phenanthryl, and anthracyl. The aromatic group may optionally contain 1-3 heteroatoms such as nitrogen, oxygen, or sulfur and can contain fused rings. Examples of aromatic group having heteroatoms include pyridyl, furanyl, pyrrolyl, thienyl, thiazolyl, oxazolyl, imidazolyl, indolyl, benzofuranyl, and benzthiazolyl. Unless otherwise noted the aromatic group may be mono- or polyvalent. In an example, the “aromatic” group may be a monocyclic aromatic group, for example a benzene group that may be unsubstituted or substituted.

As used herein, the terms “aromatic heterocycle” or “aromatic heterocyclyl” represent an aromatic cyclic group of carbon atoms wherein from one to three of the carbon atoms is/are replaced by one or more heteroatoms independently selected from nitrogen, oxygen or sulphur, e.g. having from 5-14 ring atoms. The term “aromatic heterocyclyl” is used interchangeably with ‘heteroaryl”. An aromatic heterocyclyl group may be monocyclic or polycyclic. Examples of monocyclic aromatic heterocyclyl groups (also referred to as monocyclic heteroaryl groups) include furanyl, thienyl, pyrrolyl, imidazolyl, pyridyl and pyrimidinyl. Examples of polycyclic aromatic heterocyclyl groups (also referred to as bicyclic heteroaryl groups) include benzimidazolyl, quinolinyl and indolyl. Polycyclic aromatic heterocyclyl groups include those in which only one of the rings is an aromatic heterocycle.

As used herein, the term “cyano” represents a —CN moiety.

As used herein, the term “hydroxyl” represents a —OH moiety.

As used herein, the term “alkoxy” represents an —O-alkyl group in which the alkyl group is as defined supra. Examples include methoxy, ethoxy, n-propoxy, iso-propoxy, and the different butoxy, pentoxy, hexyloxy and higher isomers.

As used herein, the term “aryloxy” represents an —O-aryl group in which the aryl group is as defined supra. Examples include, without limitation, phenoxy and naphthoxy.

As used herein, the term “carboxyl” represents a —CO₂H moiety.

As used herein, the term “nitro” represents a —NO₂ moiety.

As used herein, the term “sulfonyl” represents —S(═O)₂— moiety.

As used herein, the term “ammonium” represents ⁺NR₄ moiety.

As used herein, the term “sulfonic acid” represents —S(═O)₂—OH moiety.

The term “optionally fused” means that a group is either fused to another ring system or unfused, and “fused” refers to one or more rings that share at least two common ring atoms with one or more other rings. Fusing may be provided by one or more carbocyclic or heterocyclic rings, as defined herein, or be provided by substituents of rings being joined together to form a further ring system. The fused ring may be a 5, 6 or 7-membered ring of between 5 and 10 ring atoms in size. The fused ring may be fused to one or more other rings and may for example contain 1 to 4 rings.

The term “optionally substituted” means that a functional group is either substituted or unsubstituted, at any available position. The term “substituted” as referred to above or herein may include, but is not limited to, groups or moieties such as halogen, hydroxyl, alkyl, or haloalkyl.

The term “fluorinating” means the introduction of fluorine from a fluorinating agent to a substrate (e.g. a metal) to form a fluoride surface layer (e.g. an ex-situ SEI fluoride surface layer). Fluorinating agents are described and defined further below under the heading “Fluorinating agents”.

### Methods for Preparing SEI Layered Metal Electrodes

The methods as described herein can provide a scalable, flexible and effective method for pre-treating a metal or an electrode thereof, prior to use of the electrodes in electrochemical cells. The methods and electrodes as described herein provide for the formation of a fluoride layer on the surface of a metal or an electrode thereof, prior to cell assembly. The ex-situ treated metal electrodes provide an effective solid electrolyte interphase (SEI) layer on the electrodes in an electrochemical cell. In one embodiment or example, the SEI layer on ex-situ treated lithium metal electrodes can facilitate suppressing both high surface area lithium (HSAL) or “mossy” lithium through to dendrite growth during cycling of the cell. The methods comprise an organic solvent preparation comprising one or more fluorinating agents. The organic solvent preparation can further comprise one or more aprotic organic solvents.

In one embodiment, there is provided a method for forming an ex-situ SEI fluoride layer on the surface of a metal or an electrode thereof, the method comprising the step of contacting the surface of the metal with an organic solvent preparation.

The organic solvent preparation may comprise or consist of one or more fluorinating agents in an organic solvent. In one example, the organic solvent preparation comprises or consists of one or more aprotic organic solvents and one or more fluorinating agents. In another example, the organic solvent preparation comprises or consists of one or more aprotic organic solvents, one or more fluorinating agents, and optionally one or more additives. The one or more aprotic solvents may comprise or consist of one or more ionic liquids. The one or more additives may be selective from metal or organic salts, solvents which are SEI formers, such as vinylene carbonate (VC), fluoroethylene carbonate (FEC), and other such derivatives forming polymeric species.

In an embodiment, the methods as described herein can provide for an ex-situ treatment of a metal or an electrode thereof in forming an SEI fluoride layer on the surface of the metal, for example prior to use in an electrochemical cell. The ex-situ treated metal may also be referred to as an ex-situ SEI fluoride layered metal. Reference to “ex-situ” generally refers to formation of a SEI layer prior to exposure and cycling in an electrochemical cell environment. The ex-situ treatment of the metal or an electrode thereof facilitates formation of an effective solid electrolyte interphase (SEI) layer on the metal, which can subsequently be used in preparing an electrochemical cell. The treated “ex-situ” metal may be used in preparing a metal electrode. The prepared, pre-treated or “ex-situ” electrode may then be assembled into an electrochemical cell, for example as a next step in the method or at some later stage following storage of the electrode. In one example, the method comprises contacting the metal or electrode thereof with the organic solvent preparation before assembly into or use within an electrochemical cell.

The step of contacting the surface of the metal or electrode thereof may comprise immersing the metal or electrode thereof in the organic solvent preparation. The duration of contacting (e.g. immersing) the metal or an electrode with the organic solvent preparation may be in the range of 1 second to one week, for example 1 hour to 1 day. The duration may be at least (in hours) 1, 2, 3, 4, 5, 6, 8, 10, 12, 18, 24, 36, 48, 72, 96, 120, 144, 168, or 192. The duration may be less than (in hours) 192, 168, 144, 120, 96, 72, 48, 36, 24, 18, 12, 10, 8, 6, 5, 4, 3, 2, or 1. The duration may be provided at a range between any two of these upper and/or lower values.

The methods as described herein, or at least the step of contacting the metal with an organic solvent preparation, may be provided at a relatively low temperature such as suitable for providing an organic solvent as an effective liquid carrier for the fluorinating agent. In some examples, the temperature (in ° C.) may be less than about 150, 140, 130, 120, 110, 100, 90, 80, 70, 60, 50, 40, 30, 20, 10, 0, −10, −20, −30, −40, or −50. In some examples, the temperature (in ° C.) may be at least about −100, −90, −80, −70, −60, −50, −40, −30, −20, −10, 0, 10, 20, 30, 40, 50, 60, 70, 80, 90, or 100. The temperature may be provided in a range provided by any two of these upper and/or lower values. In some examples, the temperature (in ° C.) is provided in a range of about −100 to 150, −75 to 125, −50 to 100, −25 to 75, or 0 to 50.

The method may further comprise removing the organic solvent preparation from the surface of the metal or electrode thereof, for example a washing and/or rinsing step. The washing and/or rinsing step can be used to substantially remove the organic solvent preparation from the surface of the metal or electrode thereof, along with any remaining or residual fluorinating agent or compound thereof or additive present in the organic solvent preparation, for example. The washing and/or rinsing step may comprise removing the metal or electrode thereof from the organic solvent preparation and contacting the electrode with an organic solvent or fresh organic solvent preparation. The organic solvent may be an aprotic solvent, electrolyte or ionic liquid according to any embodiments or examples as described herein. The washing and/or rinsing step may be repeated one or more times. The washing and/or rinsing step may comprise contacting (e.g. immersing) the metal or an electrode thereof into the organic solvent (e.g. dipping the electrode into an organic solvent bath). Following the removal of the organic solvent preparation from the surface of the metal or electrode thereof, the metal or electrode thereof may be used in the preparation of an electrochemical cell or stored for later use, such as for later preparation into an electrode or assembly into an electrochemical cell.

The method may comprise a step of cleaning the surface of the metal or electrode thereof prior to the step of contacting the electrode with the organic solvent preparation. In one embodiment, the surface of the metal or electrode thereof is cleaned prior to contacting the surface of the metal with the organic solvent preparation comprising one or more fluorinating agents (i.e. precleaning). For example, the cleaning step may be used to provide a fresh surface layer of metal.

In another embodiment, there is provided a method for treating a metal or an electrode thereof prior to use in an electrochemical cell. The method can provide formation of a metal fluoride layer (e.g. SEI fluoride layer) on the surface of a metal or electrode thereof prior to exposure to an electrochemical cell environment.

In another embodiment, the method may comprise or consist of:

treating a metal or an electrode thereof according to any of the embodiments or examples thereof as described herein to form an ex-situ SEI fluoride layered metal or electrode thereof;

optionally preparing an ex-situ SEI fluoride layered metal electrode from the ex-situ SEI fluoride layered metal; and

assembling the ex-situ SEI fluoride layered metal electrode into an electrochemical cell.

Further optional steps may include cleaning, rinsing, and storage steps.

In another embodiment, the method may comprise or consist of:

optionally cleaning the surface of the metal or electrode thereof;

treating the metal or electrode thereof by contacting the surface of the metal with an organic solvent preparation comprising one or more fluorinating agents to form a metal fluoride layer on the surface of the metal or electrode thereof;

optionally rinsing the treated metal or electrode thereof;

optionally storing the treated metal or electrode thereof;

optionally configuring or assembling the treated metal or electrode thereof into an electrochemical cell.

The organic solvent preparation may comprise or consist of one or more fluorinating agents in an organic solvent. In one example, the organic solvent preparation comprises or consists of one or more aprotic organic solvents and one or more fluorinating agents. In another example, the organic solvent preparation comprises or consists of one or more aprotic organic solvents, one or more fluorinating agents, optionally one or more ionic liquids, and optionally one or more additives.

In another embodiment, there is provided a method of assembling an electrochemical cell comprising a metal electrode, whereby the steps comprise:

optionally cleaning the surface of a metal or electrode thereof;

treating the metal or electrode thereof according to any embodiments or examples of the methods as described herein to form an ex-situ SEI fluoride layered metal or electrode thereof;

optionally rinsing the ex-situ SEI fluoride layered metal or electrode thereof;

optionally storing the ex-situ SEI fluoride layered metal or electrode thereof;

configuring or assembling the ex-situ SEI fluoride layered metal or electrode thereof into an electrochemical cell.

It will be appreciated that the ex-situ SEI fluoride layered metal electrode provides a negative electrode in an electrochemical cell for association with a positive electrode and electrolyte.

The optional cleaning step may comprise cleaning the metal (e.g. foil) to remove any native “film” that may have formed on the surface of the metal. A film is typically formed from trace amounts of moisture, carbon dioxide and nitrogen that is in inert gases such as argon. In one embodiment or example, the metal is lithium which often has a native film that primarily consists of lithium oxide, lithium carbonate, lithium nitride and derivatives thereof. Depending on the exposure, the native film will not be homogeneous in thickness or character and can impact the efficacy of the pre-treatment process. Consequently, further advantages may be provided if the native film is removed by cleaning prior to the pre-treatment process. The optional cleaning step may comprise contacting the surface of the metal with an organic solvent. This can comprise brushing or wiping the surface of the metal (e.g. a foil) with the organic solvent. Any suitable organic solvent can be used to optionally clean the surface of the metal, for example pentane or THF.

In one embodiment, the organic solvent used for the optional cleaning step and the organic solvent preparation comprising the fluorinating agent may be the same. In some embodiments, the organic solvent used for the optional cleaning step may be an aprotic organic solvent. The aprotic organic solvent may be selected from an electrolyte. The aprotic organic solvent may be selected from a polar aprotic organic solvent. The aprotic organic solvent may be selected from one or more of ethers, esters, carbonates, and acetals. In an example, the aprotic organic solvent may be selected from any one or more of 1,2-dimethoxyethane, diglyme, triglyme, tetraglyme, tetrahydrofuran, and dioxolane.

In an alternative embodiment, the organic solvent used for the optional cleaning step may be a non-polar solvent, for example selected from the group consisting of pentane, hexane, benzene, chloroform, diethyl ether or 1,4 dioxane. In one embodiment, the organic solvent used for the optional cleaning step is pentane or tetrahydrofuran. The optional cleaning step may be performed using any process, for example immersion of the metal in solvent or wiping, etc. with the solvent. In one embodiment, the optional cleaning step comprising brushing the surface of the metal or electrode thereof with the organic solvent (e.g. pentane/brush or THF/brush).

Rinsing of the metal or electrode thereof after the pre-treatment process may also provide further advantages as any unreacted pre-treatment materials may interfere with the electrolyte having deleterious consequences to cell performance, via potential chemical reactions with the electrolyte or forming a passivation layer on the cathode that might impact the cyclability of the cell. Hence, in some instances, rinsing of the metal or electrode thereof may be preferred.

Storage of the ex-situ treated metal or electrodes may comprise packaging with a very low moisture/gas vapour transmission rates or in a dry room (a room with very low moisture content) in order to prevent degradation of the metal and the SEI fluoride layer. Preferably, the metal will have any native oxide layer removed or stripped, the metal then ex-situ treated according to the present disclosure, rinsed and then directly assembled into the electrochemical cell or battery to prevent degradation of the electrodes.

The metal may be provided at various purity levels, for example at least about 80%, 85%, 90%, 95%, 98%, 99%, 99.5%, 99.9%, 99.95%, or 99.99%.

The metal may be provided as a metal sheet. The metal sheet may be configured into an electrode, for example cut and rolled from the sheet into an electrode configuration. The metal sheet may be provided in a range of thicknesses depending on the application (e.g. amp hours). For example, the metal sheet may be between 1 and 1000 μm, such as between about 1 to 250 μm or between about 10 to 100 μm. It will be appreciated that the metal sheet may be provided on a supporting substrate, which may be a metal such as a copper sheet, particularly for relatively thin metal sheets such as about 100 μm or less.

### Metals

As described above, the ex-situ SEI fluoride layer may be formed by contacting the surface of the metal or electrode thereof with one or more fluorinating agents. The method for forming an ex-situ SEI fluoride layer described herein is applicable across a variety of metals, particularly those used as negative electrodes in electrochemical cells, such as primary and secondary batteries. The inventors have surprisingly identified that the presence of the ex-situ SEI layer on the metal electrode surface can lead to improved cycling and performance when used in an electrochemical cell, and the method of forming the ex-situ SEI fluoride layer is readily adaptable across various metal surfaces.

The metal may be any metal capable of reacting with a fluorinating agent to form a corresponding metal fluoride layer on the metal surface. In one embodiment, the metal is an electrochemical cell electrode, for example a metal suitable for use as a negative electrode (e.g. anode) in a battery. In one embodiment, the metal is selected from the group consisting of metals of Group 1 (e.g. lithium, potassium and sodium), Group 2 (e.g. magnesium and calcium), or Group 13 (e.g. aluminium) of the Periodic Table of Elements. In one embodiment, the metal is an alkali metal or an alkali earth metal.

In one embodiment, the metal is selected from the group consisting of lithium, magnesium, calcium, sodium, aluminium, and potassium metal. In one embodiment, the metal is selected from the group consisting of magnesium, calcium, sodium, aluminium and potassium. In one embodiment, the metal is lithium metal or magnesium metal.

In one embodiment, the metal is lithium metal. The inventors have surprisingly identified that ex-situ treatment of the surface of lithium metal or an electrode thereof with one or more fluorinating agents can produce an effective solid electrolyte interphase (SEI) layer on the lithium metal or electrodes surface that affords good cycling and performance when used in an electrochemical cell, and in some embodiments decreased dendrite formation. In contrast to the present disclosure, SEI layers generated in-situ are typically comprised of inorganic constituents (e.g. LiF, Li₂O, Li₂CO₃) closest to the native lithium metal surface and organic constituents (e.g. organic lithium salts). As some SEI constituents are more robust than others, as a result of electrolytes and additives used in the in-situ process, during the cycling there may be a steady consumption of lithium and electrolyte, which leads to a loss of capacity and a low Coulombic Efficiency (CE). Also, during cycling, the lithium electrodepositions/dissolution process may not be uniform, leading to the formation of high surface area lithium (HSAL) resulting in “dead lithium”, which no longer participates in the deposition and dissolution process. Furthermore, these dendrites can grow through the separator leading to an internal short-circuit by reaching the cathode, which can result in thermal runaway, causing serious safety problems.

In another embodiment, the metal is selected from the group consisting of magnesium, calcium and aluminium metal. In one embodiment, the metal is magnesium metal. In particular, according to some embodiments or examples, the inventors have surprisingly identified that ex-situ treatment of the surface of multivalent metals such as magnesium or electrodes thereof with one or more fluorinating agents can produce a surface SEI fluoride layer that may be ionically conductive at the metal electrolyte interface, as opposed to the native metal oxide surface layer formed in-situ if no chemical treatment was performed. With these metals, the metal oxide surface layer often leads to difficulty in ions migrating to and from the metal surface, and requires aggressive and corrosive electrolytes that are able to “strip” the oxide surface layer during in-situ electrochemical cycling. According to some embodiments or examples described herein, the inventors have surprisingly identified that the ex-situ surface treatment of multivalent metals, including magnesium metal, described herein minimises the need to use chemically aggressive electrolytes, owing to the formation of the ex-situ SEI fluoride layer at the metal electrolyte interface.

In one embodiment, there is provided a method for forming an ex-situ SET fluoride layer on the surface of lithium metal or an electrode thereof, the method comprising the step of contacting the surface of the metal with an organic solvent preparation comprising one or more fluorinating agents. In another embodiment, there is provided a method for forming an ex-situ SEI fluoride layer on the surface of magnesium metal or an electrode thereof, the method comprising the step of contacting the surface of the magnesium metal with an organic solvent preparation comprising one or more fluorinating agents. It will be appreciated that the examples and embodiments described herein in relation to methods for forming an ex-situ SEI fluoride layer on the surface of metal or an electrode thereof, and electrochemical cells (e.g. batteries) comprising SEI fluoride layered lithium metal or an electrode thereof, equally apply to other metals or electrodes thereof, and vice versa.

### Fluorinating Agents

It has been found that the fluorinating agents can effectively transfer fluorine atoms to the surface of the metals or electrodes thereof due to their reactive nature. The fluorinating agents described herein provide a source of electrophilic, nucleophilic and/or radical fluorine atoms. Briefly, nucleophilic fluorinating agents are those where the electron-rich fluorine atom (e.g. fluoride anion) serves as a reactive species. Electrophilic fluorinating agents are those where the electron-deficient fluorine atom (e.g. fluoride cation) serves as a reactive species. Unlike other fluorinated compounds that are reactively stable at room and/or higher temperatures (such as fluoropolymers Teflon, PVDF), fluoro-organics (e.g. perfluorohexane), and persistent organics pollutants (such as PFOS and PFAS,) the fluorinating agents used to prepare the SEI layer described herein can effectively transfer fluorine atoms to the metal surface under very mild conditions due to their reactive nature, to form the ex-situ SEI fluoride surface layer. In contrast to other fluorinated compounds, according to at least some examples or embodiments described herein, the fluorinating agents used to prepare the SEI layer described herein can provide one or more further advantages, including being easy and relatively safe to handle, typically solid at room temperature (e.g. the fluorinating agent may be a liquid at room temperature), enables fluorine transfer under very mild conditions, and/or is generally soluble in a variety of organic solvents (i.e. at or below room temperature). Additionally, the waste by-products from the fluorinating agent and reaction described herein may be generally organic solvent soluble and benign.

As described above, the ex-situ SEI fluoride layer may be formed by contacting the surface of the electrode with one or more fluorinating agents. The one or more fluorinating agents can be provided in an organic solvent preparation. The fluorinating agent may be selected to be soluble in suitable organic solvents, for example soluble in one or more aprotic organic solvents.

The one or more fluorinating agents may be selected from an electrophilic fluorinating agent, nucleophilic fluorinating agent, radical fluorinating agent, or any combinations thereof.

In some embodiments the fluorinating agents may provide a solid, liquid or gaseous source of fluorine. In some embodiments, the fluorinating agent may be a noble gas or an interhalogen compound. Examples of radical and/or electrophilic fluorinating agents include, but are not limited to, bromine pentafluoride, bromine trifluoride, chlorine monofluoride, chlorine pentafluoride, chlorine trifluoride, cyanuric fluoride, fluorine, iodine pentafluoride, nitrosyl fluoride, nitryl fluoride, perchloryl fluoride, sulfur tetrafluoride, trifluoromethyl hypofluorite, xenon difluoride, or xenon hexafluoride. In one example, the fluorinating agent is xenon difluoride (XeF₂).

Fluorinating agents may be selected from nitrogen containing fluorinating agents (also referred to as “N-fluorinating agent” or “N—F fluorinating agent”). For example, the nitrogen containing fluorinating agent may be an N—F fluorinating agent (also referred to as an “N—F reagent”). The N—F fluorinating agents may be selected from compounds containing an ionic NF bond (e.g. R₄N⁺F⁻) or covalent NF bond (e.g. R₂N—F or R₃N⁺—F X⁻). The N-fluorinating agents can be selected from a nucleophilic fluorinating agent (e.g. ionic NF bond, such as R₄N⁺F⁻) or an electrophilic fluorinating agent (e.g. covalent NF bond such as R₂N—F or R₃N⁺—F X⁻).

In one embodiment, the electrophilic fluorinating agent is an electrophilic N—F fluorinating agent. The electrophilic N—F fluorinating agent may be selected from a compound of Formula 1a and/or Formula 1b as described below.

In one embodiment, the fluorinating agent is a compound of Formula 1a:

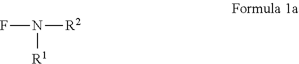

wherein

R¹ and R² are each independently selected from an optionally substituted alkyl or an electron withdrawing group, or R¹ and R² together form an optionally substituted heterocyclic ring.

In some embodiments of Formula 1a, R¹ and R² may each be independently selected from an optionally substituted electron withdrawing group or together form an optionally substituted heterocyclic ring. In one example, R¹ and R² may be an electron withdrawing group each independently selected from the group consisting of an optionally substituted sulfonyl, sulfonic acid, ammonium, nitro, cyano, halomethyl, or carboxyl. In another example, R¹ and R² may each be an optionally substituted sulfonyl group, for example a sulfonyl phenyl group. In another example, R¹ and R² may together form an optionally substituted heterocyclic ring.

In some examples, the fluorinating agent is a compound of Formula 1a selected from the group consisting of an N-fluoroarylsulfonimide such as N-fluorobenzenesulfonimide (PhSO₂)₂NF; NFSI), N-fluoroalkylsulfonamides, N-fluoro-o-benzenesulfonimide (Ph(SO₂)₂NF; NFOBS), N-fluorosultams, and N-fluorooxathiazinone dioxide. In one example, the fluorinating agent is N-fluorobenzenesulfonimide (PhSO₂)₂NF; NFBS). In another example, the fluorinating agent is N-fluoro-o-bencenesulfonimide (Ph(SO₂)₂NF; NFSI).

In one embodiment, the fluorinating agent is an ionic salt compound of Formula 1b:

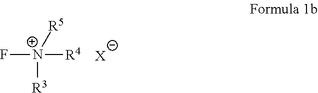

wherein

R³, R⁴ and R⁵ are each independently selected from an optionally substituted alkyl or together form an optionally substituted monocyclic or bicyclic heterocyclic ring; and

X⁻ is a counter anion.

The counter anion X⁻ may be selected from various bases, for example a hard or soft base. In some examples, the counter anion X⁻ is selected from a soft base (e.g. triflate). In some examples, the counter anion X⁻ is selected from a triflate, borate, and phosphate, each of which may contain one or more fluoro.

Fluorinating agents include N-fluoropyridinium (FPT), which shows a good solubility in a range of suitable polar organic solvents. N-Fluoro onium cations, such as N-fluoro ammonium cations, can be combined with various counter anions, such as counter anions e.g. trifluoromethanesulfonate (-OTf, triflate) to form a stable ionic compound with high melting points and suitable non-hygroscopic properties. The fluorinating power may be adjusted by the choice of the ring-substituents, where electron-withdrawing groups as substituents enhance the power. N-fluoropyridiniumtrifluoromethanesulfonate (FPT triflate) is a preferred example. FPT is relatively easy-to-handle and reproducibly affords high yields under relatively mild conditions.

In another embodiment, the fluorinating agent is an ionic salt compound of Formula 1b(i):

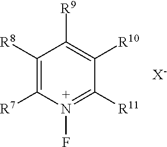

Examples of electrophilic N—F fluorinating agents of Formula 1b include, but are not limited to, N-fluoropyridinium triflate, N-fluoro-2,4,6-trimethylpyridinium triflate, N-fluoro-2,4,6-trimethylpyridinium tetrafluoroborate, N-fluoro-2,6-dichloropyridinium tetrafluoroborate, N-fluoro-2,6 dichloropyridinium triflate, N-fluoropyridinium pyridine heptafluorodiborate, N-fluoropyridinium tetrafluoroborate, N-chloromethyl-N′-fluorotriethylenediammonium bis(tetrafluoroborate) (Selectfluor®), N-chloromethyl-N′-fluorotriethylenediammonium bis(hexafluorophosphate), and N-chloromethyl-N′-fluorotriethylenediammonium bis(triflate). In some embodiments, the fluorinating agent is N-fluoropyridinium triflate.

In another embodiment, the fluorinating agent is an ionic compound of Formula 2a:

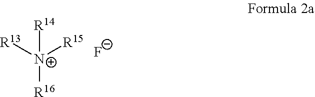

wherein
R¹³, R¹⁴, R¹⁵, and R¹⁶, are each independently selected from an optionally substituted alkyl or two or more together form an optionally substituted monocyclic or bicyclic heterocyclic ring.

In one example, an ionic compound of Formula 2a is tetrabutylammonium fluoride (TBAF).

The fluorinating agent may be provided in the organic solvent preparation at a concentration effective to control formation of the SEI fluorine layer, for example at a concentration effective to form the SEI fluorine layer over the duration of about 10 minutes to about 60 minutes. The concentration of fluorinating agent will depend on the reactivity of the particular reagent and temperature. Temperature is dependent on boiling point of solvent used. For example, a commercially available tetrahydrofuran solution of about 1M TBAF may be used.

The fluorinating agent may be provided in the organic solvent preparation at a concentration (in mol/L) of about 0.001 to about 10, about 0.01 to about 5, or about 0.1 to about 2. The fluorinating agent may be provided in the organic solvent preparation at a concentration (in mol/L) of less than about 10, 9, 8, 7, 6, 5, 4, 3, 2, 1, 0.1, 0.01, or 0.001. The fluorinating agent may be provided in the organic solvent preparation at a concentration (in mol/L) of at least about 0.0001, 0.001, 0.01, 0.1, 0.5, or 1. The fluorinating agent may be provided in the organic solvent preparation at a concentration range provided by any two of these upper and/or lower values.

In some embodiments, it has been found that the fluorinating agents can effectively transfer a high amount of fluorine atoms to the surface of the metals or electrodes, resulting in the formation an ex-situ SEI fluoride rich layer. As used herein, the term “fluoride rich” refers to a SEI fluoride layer comprising a high amount of fluoride compared to the surface of non-surface treated metals, which can be determined by X-ray photoelectron spectroscopy (XPS). According to some embodiments or examples, XPS analysis can also be used to determine a ratio metal:fluorine (e.g. Li/F ratio or Mg/F ratio) in the ex-situ SEI fluoride layer on the surface of the metal following treatment with fluorinating agents. A low metal:fluorine ratio points to the presence of fluorine due to presence of the ex-situ treated metal surface layer. Conversely, a high metal:fluorine ratio can identify a surface layer on the metal without enrichment of fluorine, e.g. on metal surfaces not treated with fluorinating agents. An example of a low metal:fluorine ratio according to at least some embodiments or examples described herein may be 2.0 or less,

In some embodiments, the ex-situ SEI fluoride layer has a metal:fluorine ratio of less than about 10, 9, 8, 7, 6, 5, 4.5, 4, 3.5, 3, 2.5, 2, 1.8, 1.6, 1.4, 1.2, 0.8, 0.5. The ex-situ SEI fluoride layer may have a metal:fluorine ratio provided at a range between any two of these upper and/or lower values, for examples between about 1.0 to about 2.0. Byway of explanation only, non-fluorinated surfaces as described herein generally exhibit very high metal:fluorine ratios when measured using XPS, for example at least about 14 and sometimes as high as about 50 or more. This highlights a lack of enrichment of fluorine on the surface of the metals not treated with a fluorinating agent as described herein.

According to some embodiments or examples described herein, the ex-situ treatment of metals or electrodes thereof fluorinating agents described herein can result in at least a 2, 4, 6, 8, 10, 12, 15, 20, 30, 40 or 50 fold decrease in the metal:fluorine ratio compared to a non-treated (e.g. no fluorinating agent or treated with a non-fluorinating agent (e.g. perfluorohexane)) metal or electrode thereof. It will be appreciated that such a decrease in the metal:fluorine ratio following ex-situ treatment with fluorinating agents compared to non-fluorinated metal electrodes points to an increase in fluorine enrichment and the formation of metal fluoride (e.g. LiF) as a result of the ex-situ SEI fluoride layer. Protocols to measure and obtain XPS spectra are well known, and further outlined in the Examples.

The ex-situ SEI fluoride layer may have a thickness (i.e. a cross-sectional distance measuring across the ex-situ SEI fluoride layer). In some embodiments the thickness of the ex-situ SEI fluoride layer may be at least about 0.1, 0.2, 0.5, 0.8, 1, 1.5, 2, 2.5, 3, 4, 5, 7, 10, 15, 20, 30, 40, 50, 75, 100, 150, 200, 500 or 1,000 nm. In some embodiments, the thickness of the ex-situ SEI fluoride layer may be less than about 1,000, 500, 200, 150, 100, 75, 50, 40, 30, 20, 15, 10, 7, 5, 4, 3, 2.5, 2, 1.5, 1, 0.8, 0.5, 0.2 or 0.1 nm. The ex-situ SEI layer thickness may be provided at a range between any two of these upper and/or lower values, for examples between about 1 nm to about 50 nm. The thickness of the ex-situ SEI fluoride layer can depend on the contact time (e.g. immersion time) of the metal with the solvent comprising the fluorinating agent. The thickness can also be tuned depending on the properties of the surface layer, for example SEI layers that are insulating may be thinner whereas conductive SEI layers may be thicker. The thickness can be measured using atomic force microscopy. Due to the difference in mechanical properties, the tip of the AFM can differentiate between the ex-situ SEI layer and underlying metal and determine the thickness of the ex-situ SEI layer.

### Organic Solvent Preparation

Formation of an SEI fluoride layer on a metal or metal electrodes can be achieved by the ex-situ methods as described herein using organic solvent preparations that comprise various fluorinating agents and optional additives. The various fluorinating agents and optional additives can be selected from those not suitable for direct use within battery electrolyte solutions. In other words, one or more fluorinating agents and optional additives may be used in the present ex-situ methods that are selected from those that are suitable and/or those that are unsuitable for use in any in-situ methods or battery electrolyte solutions, such as operation or use in forming an initial SEI layer within a secondary battery environment. The ex-situ methods as described herein can therefore enable access to a broad range of fluorinating agents and optional additives in preparing distinctive SEI layers that can be tuned to provide particular compositions and properties, and which may not otherwise be accessed through in-situ methods.

In addition to the one or more fluorinating agents, the organic solvent preparation may comprise an aprotic organic solvent. The aprotic organic solvent may be selected from an electrolyte. The aprotic organic solvent may be selected from a polar aprotic organic solvent. The aprotic organic solvent may be selected from one or more of ethers, esters, carbonates, and acetals. In an example, the aprotic organic solvent may be selected from any one or more of 1,2-dimethoxyethane, diglyme, triglyme, tetraglyme, tetrahydrofuran, and dioxolane.

The aprotic organic solvent may comprise or further consist of one or more ionic liquids. The one or more ionic liquids may be selected from one or more of 1-butyl-1-methylpyrrolidinium-cation (Pyr₁₄+), and 1-propyl-1-methylpyrrolidinium and bis(fluorosulfonyl)imide (Pyr₁₃FSI).

The organic solvent preparation may comprise an optional additive. For example, the organic solvent preparation may further comprise or consist of one or more additives. The one or more additives may be selected from one or more alkali metal salts, for example lithium metal or organic soluble salts (e.g. tetrabutylammonium nitrate (TBANO₃). The alkali metal salts can be added in the ex-situ preparation to further enhance the conductivity by improving the ion mobility of the electrolyte following preparation of the electrode and incorporation into an electrochemical cell, for example a secondary lithium battery. The alkali metal salts may be lithium metal salts. The alkali metal salts may be selected from one or more of LiPF₆ LiBF₄, LiAsF₆, LiSbF₆, LiClO₄, LiC(SO₂CF₃)₃, LiN(SO₂CF₃)₂, LiSCN, LiSO₂CF₂CF₃, LiC₆F₅SO₃, LiO₂CCF₃, LiSO₃F, LiB(C₆H₅)₄, LiCF₃SO₃, and LiNO₃. In one example, the additive is LiNO₃. It will be appreciated that the additives can be selected to provide one or more properties including: dissolving and dissociating in the organic solvent preparation, being chemically stable in the electrochemical environment, supporting the metal fluoride (e.g. LiF of MgF₂) or SEI formation on the metal electrode, being of low toxicity, and/or environmentally-friendly. In an example, the lithium metal salts are selected from LiPF₆, and LiClO₄. In one example of an additive mixture, there is provided LiTFSI as a conductive salt and LiNO₃ as an additional additive.

In addition to the fluorinating agent, the organic solvent preparation may comprise or consist of a combination of aprotic organic solvents, optionally with one or more ionic liquids, and optionally with one or more additives. In one example, the organic solvent preparation comprises or further consists of one or more of Pyr₁₄TFSI, TEGDME, DME, DOL, LiTFSI, TBANO₃, and LiNO₃.

### Electrode

It will be appreciated that the metal electrode has a surface that comprises the metal. For example, a lithium metal electrode has a surface that comprises lithium metal. In another example, a magnesium metal electrode has a surface that comprises magnesium metal. Typically a metal scaffold is provided with a surface comprising a metal composition. In some embodiments, the metal electrode is used as a negative electrode. In one embodiment, the metal species is lithium metal. In another embodiment, the metal species is magnesium metal.

It will be appreciated that the metal electrode may be provided in any shape, size, thickness, or configuration. For example, the “electrode” may simply be a strip or section of metal. The electrode may be retained in the same shape, size, thickness, or configuration, after pre-treatment, or further modified. The metal electrode may comprise a current collector (e.g. copper) coated with the metal. In some embodiments the metal may be provided on one side of a planner electrode (e.g. lithium coated onto a thin Cu current collector to form a lithium metal electrode). In other embodiments, the metal electrode may not have a current collector. The metal may be coated on both faces of the electrode. In one embodiment, the metal electrode is a lithium metal electrode. The lithium metal electrode may comprise or consist of lithium metal (e.g. lithium metal foil). In another embodiment, the metal electrode is a magnesium metal electrode. The magnesium metal electrode may comprise or consist of magnesium metal (e.g. magnesium metal foil).

### Electrochemical Cells

It will be appreciated that an electrochemical cell comprises a negative electrode and a positive electrode in fluidic communication with an electrolyte. A membrane is typically provided between the electrodes. The negative electrode can be a metal electrode as described herein. The positive electrode can comprise a positive electrode active material.

In one embodiment, there is provided an electrochemical cell comprising:

a negative electrode provided by an ex-situ treated metal electrode according to any embodiments or examples as described herein;

a positive electrode comprising a positive electrode active material; and

an electrolyte comprising one or more electrolyte solvents.

In one embodiment, the electrochemical cell is a secondary battery.

### Secondary Battery

The general components of a secondary battery are well known and understood in the art of the invention. The principal components are:

a battery case of any suitable shape, standard or otherwise, which is made from an appropriate material for containing the electrolyte, such as aluminium or steel, and usually not plastic;

battery terminals of a typical configuration;

a negative electrode;

a positive electrode;

a separator for separating the negative electrode from the positive electrode; and an electrolyte.

The negative electrode comprises a metal substrate, which acts as a current collector, and a negative electrode material. The metal can be deposited onto/into any of these materials electrochemically in the device. In one embodiment or example, the secondary battery is a secondary lithium battery, and the negative electrode material is a lithium metal or a lithium alloy forming material.

The metal substrate underlying the metal (e.g. lithium) can be of importance in determining the cycle performance of the cell. This element may also have the role of current collector in the cell. The metal substrate may be any suitable metal or alloy, and may for instance be formed from one or more of the metals Pt, Au, Ti, Al, W, Cu or Ni. In one example, the metal substrate is Cu or Ni.

In the context of the present disclosure, the negative electrode surface (i.e. the lithium electrode) has a metal fluoride SEI layer on the surface from an ex-situ pre-treatment with one or more fluorinating agents, prior to incorporation into the secondary battery.

The positive electrode may be formed from any typical lithium intercalation material, such as a transition metal oxides and their lithium compounds. As known in the art, transition metal oxide composite material is mixed with a binder such as a polymeric binder, and any appropriate conductive additives such as conductive carbons (e.g. graphite), before being applied to or formed into a current collector of appropriate shape.

Types of batteries may include Lithium-Sulfur and Lithium-Air batteries, for example.

Any typical separator known in the art may be used, including glass fibre separators and polymeric separators, particularly microporous polyolefins.

Usually the battery will be in the form of a single cell, although multiple cells are possible. The cell or cells may be in plate or spiral form, or any other form. The negative electrode and positive electrode are in electrical connection with the battery terminals.

A method of assembling an electrochemical cell comprising the metal electrode described herein may comprise any one or more of the following steps. The surface of the metal electrode may be optionally cleaned to provide a fresh metal surface. The metal electrode can be ex-situ treated according to any of the embodiments or examples as described herein. The ex-situ treated metal electrode may be washed and/or rinsed with an organic solvent as described herein. The ex-situ treated metal electrode may be stored before use in an electrochemical cell, for example for a duration from minutes to months. The ex-situ treated metal electrode can then be assembled into an electrochemical cell to provide a negative electrode. The assembly provides the negative electrode in combination with a positive electrode and electrolyte to enable formation of an effective solid electrolyte interphase (SEI) layer on the negative electrode.

### Electrolyte Solvents

The one or more electrolyte solvents may be selected from ethers, esters, carbonates, and acetals. In one example, the one or more electrolyte solvents are selected from 1,2-dimethoxyethane, diglyme, triglyme, tetraglyme, ethylene carbonate, propylene carbonate, dimethyl carbonate, tetrahydrofuran, and dioxolane.

The electrolyte may comprise one or more alkali metal salts. The alkali metal salts may be selected from the group consisting of LiPF₆ LiBF₄, LiAsF₆, LiSbF₆, LiClO₄, LiAlCl₄, LiGaCl₄, LiC(SO₂CF₃)₃, LiN(SO₂CF₃)₂, LiSCN, LiSO₂CF₂CF₃, LiC₆F₅SO₃, LiO₂CCF₃, LiSO₃F, LiB(C₆H₅)₄, LiCF₃SO₃, and mixtures thereof.

The electrolyte may comprise one or more ionic liquids. In one example, the one or more ionic liquids are selected from 1-butyl-1-methylpyrrolidinium-cation (Pyr₁₄+) and 1-propyl-1-methylpyrrolidinium bis(fluorosulfonyl)imide (Pyr₁₃FSI).

The electrolyte may comprise an additive selected from one or more alkali metal salts of LiPF₆ LiBF₄, LiAsF₆, LiSbF₆, LiClO₄, LiAlCl₄, LiGaCl₄, LiC(SO₂CF₃)₃, LiN(SO₂CF₃)₂, LiSCN, LiSO₂CF₂CF₃, LiC₆F₅SO₃, LiO₂CCF₃, LiSO₃F, LiB(C₆H₅)₄, LiCF₃SO₃, LiNO₃, and mixtures thereof.

Electrolyte additives can further improve certain characteristics of the cell. Depending on the type of additive, it may increase the safety of a battery due to non-flammability or it supports SEI formation on the metal anode. For example, the use of LiNO₃ in electrolytes significantly improves cycling efficiency, since LiNO₃ participates in the formation of a stable SEI on the lithium metal surface preventing reactions with polysulfides and suppressing the polysulfide shuttle. Although LiNO₃ may not be as beneficial on the cathode side, it is still a standard additive due to the advantages on the anode side. Generally, additives are added in very small amounts (<5 wt %), however, it has to be considered that not only the initial SEI formation consumes the additive but there is also a trace consumption in each cycle. Therefore, the beneficial effect of additives is only present until they are completely consumed.

### Negative Electrode

The negative electrode (or anode) comprises a metal substrate, which acts as a current collector, and a negative electrode material. In some embodiments, the negative electrode material comprises or consists of an ex-situ SEI fluoride layered metal described herein. The metal can be deposited onto/into any of these materials electrochemically in the device.

The underlying metal substrate can be of importance in determining the cycle performance of the cell. This element may also have the role of current collector in the cell. The metal substrate may be any suitable metal or alloy, and may for instance be formed from one or more of the metals Pt, Au, Ti, Al, W, Cu or Ni. In one example, the metal substrate is Cu or Ni.

The negative electrode can further comprise a negative electrode active material selected from the group consisting of coke, carbon black, graphite, acetylene black, carbon fibers, glassy carbon, meso carbon microbeads, lithium silicon, or lithium tin composites and alloys and mixtures thereof.

In some embodiments lithium metal negative electrodes or anodes are often combined with a sulfur cathode.

### Positive Electrode

The positive electrode active material may be selected from sulfur, nickel, magnesium cobalt cathode material, for example. In one example, the positive electrode active material comprises a sulfur containing material.

The positive electrode active material may be mixed with a conductive additive. For example, a conductive additive selected from the group consisting of acetylene black, carbon black, conducting polymers, graphite, nickel powder, aluminium powder, titanium powder, stainless steel powder, and mixtures thereof.

The positive electrode active material may be selected from the group consisting of lithiated oxides, lithiated sulfides, lithiated selenides and lithiated tellurides of the group selected from vanadium, titanium, chromium, copper, molybdenum, niobium, iron, iron phosphate, nickel, cobalt, manganese, and mixtures thereof.

Sulfur positive electrodes (or cathode) usually comprises sulfur mixed in a carbon matrix on an aluminium current collector. The carbon matrix provides conductivity and facilitates volume change (e.g. up to about 80%) during cycling. Suitable carbon materials may include microporous, mesoporous and macroporous carbons as well as graphene and activated carbon.

### EXAMPLES

The present disclosure is further described by the following examples. It is to be understood that the following description is for the purpose of describing particular embodiments only and is not intended to be limiting with respect to the above description.

### Materials and Methods

All chemicals were used as received, except lithium bis(trifluoromethanesulfonyl)imide (3M, LiTFSI) which was dried under reduced pressure at 80° C. for 48 h and Solupor® Membrane 7P03A (Lydall) which was dried at 40° C. under reduced pressure for 4

NMR experiments were performed on a Bruker Avance 400 MHz NMR spectrometer with a 5 mm broadband probe (400.13 MHz ¹H frequency). NMR experiments were performed with the sample held at 25±0.1° C. Chemical shifts for all experiments are referenced using the Unified Scale relative to 0.3% tetramethylsilane in deuteriochloroform.

X-ray photoelectron spectroscopy (XPS) analysis was performed using either an AXIS Nova or an AXIS Ultra-DLD spectrometer (Kratos Analytical Inc., Manchester, UK) with a monochromated Al K_{α} source at a power of 180 W (15 kV×12 mA) and a hemispherical analyser operating in the fixed analyser transmission mode. The total pressure in the main vacuum chamber during analysis was typically between 10⁻⁹ and 10⁻⁸ mbar. Survey spectra were acquired at a pass energy of 160 eV and step size 0.5 eV. To obtain more detailed information about chemical structure, oxidation states etc., high resolution spectra were recorded from individual peaks at 40 eV or 20 eV pass energy and step size 0.1 eV, typically yielding a FWHM of <0.8 eV for the Ag 3d_{5/2} peak and <0.85 eV for the ester peak in PET during performance tests. The samples were analysed at a nominal photoelectron emission angle of 0° w.r.t. the surface normal. The sampling depth varied from between 0 nm to approx. 10 nm.

### Example 1: Preparation of Solid Electrolyte Interface (SEI) on Lithium Metal Electrodes

Lithium metal disks (as supplied by Gelon New Battery Materials Co., Ltd.) were washed with organic solvent (e.g. 1,2-dimethoxyethane (DME), and the solvent was removed by evaporation before use.

Electrolyte and coin cell preparation, immersion of the electrodes as well as sample preparation for XPS and nuclear magnetic resonance spectroscopy (NMR) were carried out in an argon-filled glovebox (Korea Kiyon) with H₂O values below 2 ppm and O₂ values below 1 ppm.

Sulfur ((sulfur nanoparticles/nanopowder (S, 99.99%, <55 nm) (SkySpring Nanomaterials, Inc.) or sulfur (Sigma Aldrich) was mixed with carbon and roll-milled in a ball-milling jar with zirconia balls for one day. Then the binder (Kynar Flex® 2801 PVdF (Arkema) or KF Polymer PVdF (Kureha) dissolved in N-methyl-2-pyrrolidone (Sigma Aldrich, NMP) was added. Additional NMP was added until the slurry had an appropriate consistency. After roll-milling for another day the slurry was ball-milled for 3 h for LITXcarbons and 6 h for the other carbons at a speed of 400 rpm.

In order to compare the effect of melt diffusion, additional electrodes were prepared by this method in which sulfur and carbon were mixed and then stored in an oven at 155° C. for 12 h under nitrogen atmosphere. Afterwards the electrode slurry was prepared with the method detailed above. The cathode coatings of different thicknesses were prepared using either doctor blade or k-bar. The slurries were coated onto carbon-etched aluminium foil (Kawatake Electronics Co., Ltd.) (20 μm thickness) and dried for 18 h under reduced pressure at 60° C., then the electrodes (12.7 mm diameter) were punched out, dried for another 24 h under vacuum and weighed.

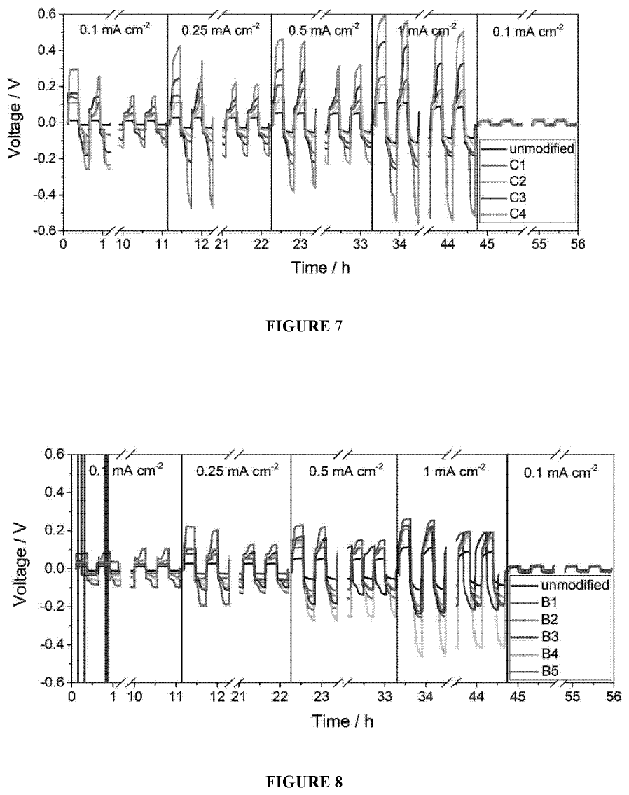

Butyl-1-methylpyrrolidinium bis(trifluoromethanesulfonyl)imide (Iolitec Ionic Liquid Technologies, Pyr₁₄TFSI) and tetraethylene glycol dimethyl ether (Sigma Aldrich, TEGDME) were mixed 1:1 (per volume) and 1 M lithium bis(trifluoromethanesulfonyl)imide (3M, LiTFSI) and 0.1 M lithium nitrate (Sigma Aldrich, LiNO₃) were added. Before use, the electrolyte was stirred over night to ensure the salts were completely dissolved. For comparison a commercial organic electrolyte (1,2-dimethoxyethane (DME): 1,3-dioxolane (DOL) 1:1, 1 M LiTFSI, 0.2 M LiNO₃, Novolyte Technologies) was used. For electrochemical cell testing coin cells CR2032 were assembled in a two-electrode setup (FIG. 7). For long-term full cell cycling lithium disks (Gelon New Battery Materials Co., Ltd.) (12 mm diameter, 400 μm thickness) as counter electrode und sulfur cathodes (12.7 mm diameter) were used as working electrode.

The electrodes were separated by Solupor® Membrane 7P03A (Lydall) (16 mm diameter, 50 μm thickness) wetted with 40 μL electrolyte (Pyr₁₄TFSI:TEGDME 1:1, 1 M LiTFSI, 0.1 M LiNO₃ or DME:DOL 1:1, 1 M LiTFSI, 0.2 M LiNO₃). For overvoltage evolution and EIS, lithium disks were used as counter and working electrodes. For these experiments the Solupor® Membrane 7P03A was only wetted with 30 μL of the same electrolytes.

As described above, the lithium metal electrodes were washed with DME, immersed for different time periods in the solvent mixtures presented in Table 1 below, again washed in DME to remove an excess of chemicals used for pre-immersion and then the solvent was evaporated prior to cell assembly. Structures of some of these chemicals are presented in Scheme 1 below. The mixtures were prepared by dissolving the salts in the solvents. The solution for C3 and C4 was prepared by adding Pyr₁₃FSI to the commercial 1 M TBAF in THF solution and then evaporating the THF under reduced pressure.

TABLE 1
Solvents and salts used for immersion of lithium metal electrodes.

| | | | | Immersion/ | |
|---|---|---|---|---|---|
| Name | Solvent | Salt | Concentration | Contact time | |
| A1 | Pyr₁₃FSI | — | — | 2 | d |
| A2 | Pyr₁₃FSI | — | — | 7 | d |
| A3 | Pyr₁₃FSI | LiNO₃ | 0.05M | 2 | d |
| A4 | Pyr₁₃FSI | LiNO₃ | 0.05M | 7 | d |
| B1 | THF | FPT | Saturated Solution | 2 | d |
| B2 | THF | FPT | Saturated Solution | 5 | d |
| B3 | THF | FPT | Saturated Solution | 7 | d |
| B4 | TEGDME | FPT | 0.5M | 2 | d |
| B5 | Pyr₁₃FSI | FPT | 0.5M | 2 | d |
| C1 | THF | TBAF | 1M | 3 | hr |
| C2 | THF | TBAF | 1M | 5 | hr |
| C3 | Pyr₁₃FSI | TBAF | 1M | 1 | d |
| C4 | Pyr₁₃FSI | TBAF | 1M | 2 | d |
| D1 | THF | TBAF and FPT | 1M and 0.5M | 5 | s |

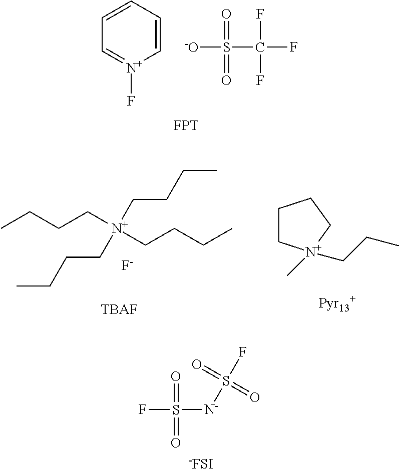

All cells were tested either at 50° C. (IL-based electrolyte) or at room temperature (commercial organic electrolyte).

### Example 2: Long-Term Cycling

For long-term cycling a MACCOR battery test system (MACCOR Series 4000, MACCOR INC., Tulsa, Okla., USA) was used. A C-rate of 0.05 C was applied for one cycle after 12 h of resting under open circuit voltage (OCV) conditions. Then the C-rate was increased to 0.1 C for 20 cycles. In order to investigate not only the cycling behavior but also the self-discharge, the cells were rested for one day afterwards and cycled again for 30 cycles at 0.1 C. This scheme was repeated until the cell reached 200 cycles. The cells with ex-situ surface treated lithium were cycled at a C-rate of 0.05 C in the first cycle after a rest period of 12 h under OCV conditions and then at 0.1 C for 199 cycles.

Electrochemical Impedance Spectroscopy EIS measurements were performed with symmetrical Li∥Li cells using a BioLogic VMP III potentiostat in a frequency range between 0.1 M Hz and 0.1 Hz and an amplitude of 10 mV. One hour after cell assembly the impedance was measured, then the cells were cycled at a current density of 0.1 mA cm⁻² for 20 cycles by applying 16 minutes nominally positive current followed by 16 minutes of current of the opposite polarity in each cycle. Afterwards the impedance was measured again. Then the same procedure was repeated with current densities of 0.25 mA cm⁻², 0.5 mA cm⁻², 1.0 mA cm⁻² and then again with 0.1 mA cm⁻². After a rest period of 24 h the whole procedure was repeated.

The abbreviations shown in the graphs are explained in Table 2 below. The first impedance of each measurement is defined as electrolyte resistance, whereas the interfacial resistance is determined by finding the minimum of the impedance in the Nyquist plot after the first semi-circle and subtracting the electrolyte resistance from this value.

TABLE 2
Explanation of measurement abbreviations for impedance
measurements after different cycling times.

| | Cycling | |
|---|---|---|
| Measurement | time/hr | Condition |
| 1 | 0 | After 1 hr of rest |
| 2 | 12 | 1 + 20 cycles at 0.1 mAh g⁻¹ |
| 3 | 24 | 2 + 20 cycles at 0.25 mAh g⁻¹ |
| 4 | 36 | 3 + 20 cycles at 0.5 mAh g⁻¹ |
| 5 | 48 | 4 + 20 cycles at 1 mAh g⁻¹ |
| 6 | 60 | 5 + 20 cycles at 0.1 mAh g⁻¹ |
| 7 | 60 | 6 + 24 h rest |
| 8 | 72 | 7 + 20 cycles at 0.1 mAh g⁻¹ |
| 9 | 84 | 8 + 20 cycles at 0.25 mAh g⁻¹ |
| 10 | 96 | 9 + 20 cycles at 0.5 mAh g⁻¹ |
| 11 | 108 | 10 + 20 cycles at 1 mAh g⁻¹ |
| 12 | 120 | 11 + 20 cycles at 0.1 mAh g⁻¹ |

### Example 3: No Immersion of Electrode in Fluorinating Agent

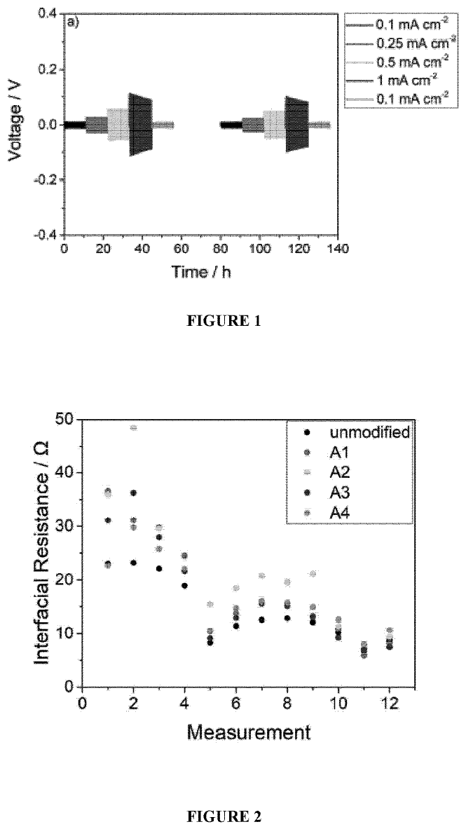

The cell using non-surface treated lithium metal electrodes (i.e. no immersion of electrode in fluorinating agent) shows stable and smooth overvoltage, which increases when the current density is increased (FIG. 1). At a current density of 0.1 mA cm⁻² the overvoltage is ±0.01 V, at 0.25 mA cm⁻² it is +0.03 V and at 0.5 mA cm² the overvoltage reaches ±0.05 V.

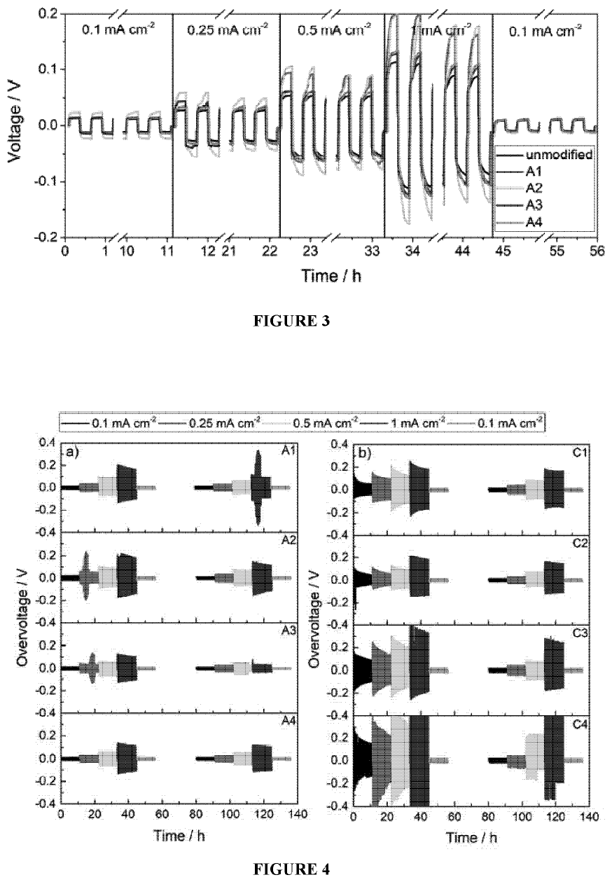

FIG. 2 shows the interfacial resistance during cycling of an ionic liquid (IL) based electrolyte with and without LiNO₃ additive. FIG. 3 shows the overvoltage for the same cells with and without LiNO₃ additive.

### Example 4: Immersion of Electrode in Fluorinating Agent

The fluorinating agents, TBAF and FPT, and a combination of the two were investigated. The electrodes were immersed for five hours and then applied in cells. The overvoltage evolution and the interfacial resistance are presented in FIGS. 4, 5 and 6. Compared to cells without the fluorinating agents (e.g. FIGS. 1 to 3), the overvoltages are comparable although higher voltages after C-rates of 120 are observed for the TBAF/IL systems.

### Example 5: Investigation of Different Electrolytes/Solvents Used to Dissolve the Fluorinating Agent

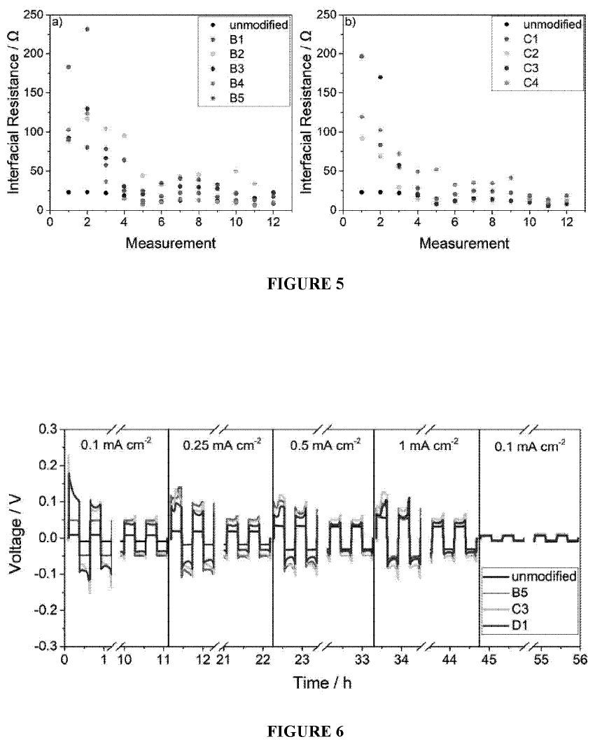

The results using the IL-based electrolyte are compared with the results using different organic electrolytes, see FIGS. 4(b), 5, 6, 7 and 8. Immersion in TBAF/THF (C1) increases the over voltage compared to unmodified lithium metal (±0.03 V in the initial cycles), but the general shape is still the same. All the cells with electrodes surface treated with fluorinating agents show very high initial interfacial resistances in the organic electrolyte. The cycling behaviour of FPT is further improved if it is dissolved in IL. Additionally, the combination of both fluorine sources in THF provides further advantages, see FIGS. 6 and 7.

### Example 6: Influence Fluorinating Agents have on Specific Discharge Capacity

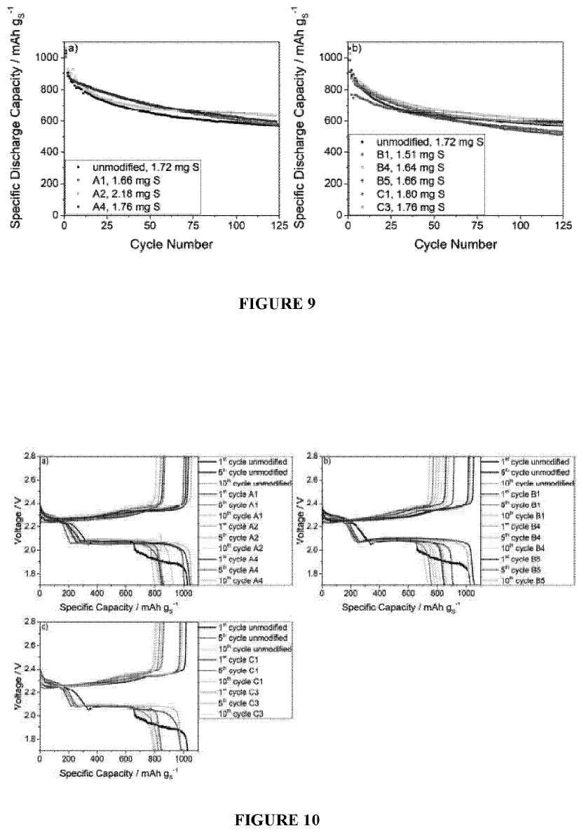

FIGS. 9 and 10 show trends of the specific discharge capacity with and without fluorinating agents when used in a lithium-sulfur battery.

After 100 cycles, the specific discharge capacities of the cells are between 539 and 649 mAh gS⁻¹. All cells show defined voltage plateaus for discharge (2.3 V and 2.1 V) and charge (between 2.3 and 2.4 V), although the first discharge plateau is relatively short.

Pre-immersion of lithium metal electrodes showed beneficial properties, which in the above examples were tested in combination with sulfur cathodes. For FPT a shorter immersion time (e.g. about 2 days) was effective. It was also observed that the change from THF to TEGDME as solvent further improves the performance. The choice of solvent influenced the composition of the artificial SEI or at least the upper organic layer of the SEI.

### Example 7: Comparison of Precleaning Electrode Prior to Immersion Step

The differences in the electrodes with or without precleaning was investigated using commercial as-supplied Li-electrodes that were precleaned with various solvents. Briefly, precleaning the Li-electrode surface to remove native material was performed using either a pentane/brush (Pentane-cleaned) or a THF/brush (THF-cleaned) on commercially supplied electrodes. The cleaned Li electrodes were compared to as-supplied commercial Li-electrodes.

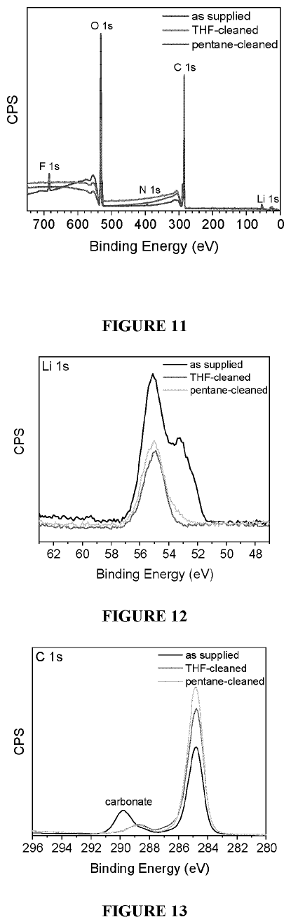

From the survey XPS spectra of FIGS. 11, 12 and 13, the as-supplied Li electrode contains a trace amount of nitrogen, some fluorine, carbon and a significant amount of oxygen material. Both pentane and THF cleaning methods decrease the amount of oxygen and nitrogen present. Removal of both lithium oxide and carbonates from lithium electrode is also evident with THF cleaning being slightly better. The high-resolution carbon spectra of FIG. 13 show a reduction of carbonate while an increase in total carbon with both pentane and THF cleaning processes and the high-resolution lithium spectra of FIG. 12 show a less complex lithium surface after cleaning due to the removal of species such as lithium carbonate. Table 3 summarises the atomic % of various elements present on the surface of the lithium metal with or without precleaning electrode prior to immersion as shown in FIGS. 11, 12 and 13.

TABLE 3
Elemental quantification (Atomic %) for lithium electrode as supplied (A), lithium
electrode cleaned in pentane (B) and lithium electrode cleaned in THF (C).

| | | | | | | | Li/O | Li/CO₃²⁻ |
|---|---|---|---|---|---|---|---|---|
| | | | | | | | comparison | comparison |
| | | | | | Li/O | Li/CO₃²⁻ | (control/ | (cleaned/ |
| # | Conditions | % Li | % O | % CO₃²⁻ | ratio | ratio | cleaned) | control) |
| A | Li as | 27.1 | 29.5 | 8.3 | 0.9 | 3.3 | — | — |
| | supplied | | | | | | | |
| B | Li cleaned | 12.8 | 14.5 | 0.8 | 0.9 | 16.0 | 1 | 4.8 |
| | in pentane | | | | | | | |
| C | Li cleaned | 11.4 | 17.4 | 0.6 | 0.7 | 19.0 | 1.3 | 5.8 |
| | in THF | | | | | | | |

Li metal as supplied (control; Sample A) comprised a surface having a relatively low Li/CO₃²⁻ ratio of 3.3, highlighting the presence of carbonate anions on the surface. Compared to the lithium metal as-supplied (control; Sample A), cleaning with pentane/brush (Sample B) and cleaning with THF/brush (C) results in significantly higher Li/CO₃²⁻ ratios. A higher Li/CO₃²⁻ ratio indicates a lower presence of CO₃²⁻ on the surface. When compared with the lithium metal as-supplied (control; Sample A), cleaning with pentane/brush (Sample B) and cleaning with THF/brush (Sample C) results in ˜5 and ˜6-fold increase in the Li/CO₃²⁻ ratio, respectively, highlighting the removal of native surface species such as lithium carbonate from the metal surface.

### Example 8: Comparison of Precleaned Electrode after Immersion with Fluorinating Agents

The electrode surface was precleaned to remove native material using a THF/brush on commercially supplied lithium electrode. The cleaned Li electrode was then immersed in a TBAF/THF (0.5M THF) for 20 minutes. A Li electrode cleaned with THF/brush without immersion in TBAF/THF was measured as a control.

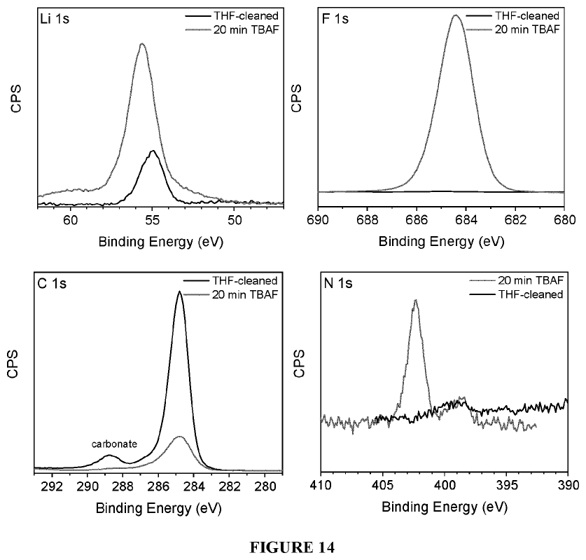

From the high level XPS spectra of FIG. 14, it is evident that after 20 minutes, the pre-cleaned Li and TBAF in THF sample shows a dramatic increase in fluorine and lithium, that can be attributed to the formation of lithium fluoride. There is also the introduction of nitrogen. Similarly analysis of the carbon, oxygen and nitrogen XPS spectra show changes over this 20 minute exposure time. TBAF in THF on precleaned Li electrodes results in removal of carbonate groups and hydrocarbon in the high-resolution carbon spectra. The fluorine spectra shows fluorine in the form of fluoride on the lithium electrode, thus, together with the survey data, suggests the formation of a significant amount of lithium fluoride. Table 4 summarises the atomic % of various elements present on the surface of the lithium metal following cleaning in THF and subsequent immersion with fluorinating agent as shown in FIG. 14.

TABLE 4
Elemental quantification (Atomic %) for lithium electrode
cleaned in THF (C) and lithium electrode cleaned
in THF, then immersed in TBAF in THF (D).

| | | | | Li/F | Li/F comparison |
|---|---|---|---|---|---|
| # | Conditions | % Li | % F | ratio | (control/F agent) |
| C | Li cleaned in THF | 11.4 | 0.8 | 14.1 | — |
| D | Li cleaned in THF, | 40.6 | 39.6 | 1.0 | 14.1 |
| | treated with TBAF in | | | | |
| | THF (20 minutes) | | | | |

Li metal cleaned with THF without immersion in fluorinating agent (control; Sample C) comprised a surface with a high Li/F ratio, highlighting the lack of fluorine enrichment on the surface. In contrast, compared to the lithium metal cleaned in THF/brush (control; Sample C), the chemical treatment of cleaned lithium with TBAF in THF (Sample D) results in an 14-fold decrease of the Li/F ratio, which points to a significant increase in the amount of fluorine present in the in ex-situ treated metal surface layer, as a result of the formation of the ex-situ SEI fluoride layer.

### Example 9: Comparison of as-Supplied Electrode (i.e. No Precleaning) after Immersion with Fluorinating Agents

In the next set of experiments, Li-electrodes were used without any precleaning, and immersion with solvent and fluorinating agent occurred outside a glovebox.

Li and THF only (Sample 10A) and Li and TBAF/TBANO₃ in THF (Sample 11A) were all used with the same metal to reagent mole ratio and 20 minute immersion/contact time to the solutions (e.g. solvent only or solvent/fluorinating agent). All samples once treated were subsequently washed and prepared for XPS analysis as per previous experiments.

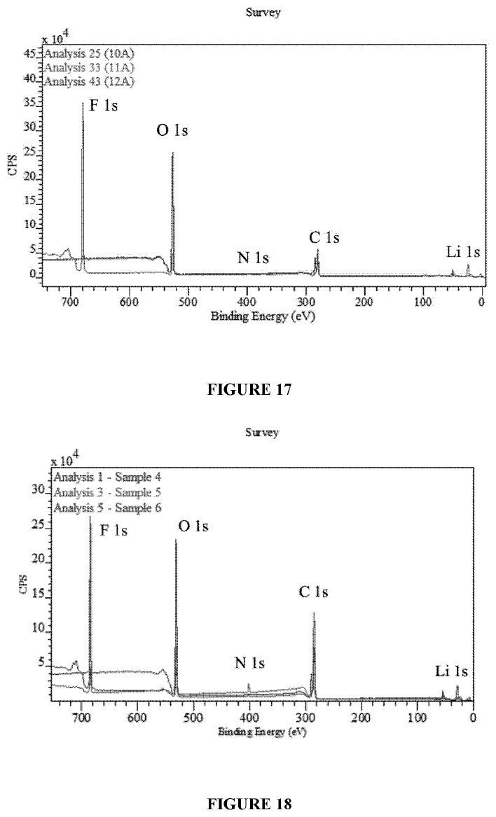

FIG. 17 compares the effect chemical treatments of as-supplied lithium and THF (Sample 10A), and as-supplied Li and TBAF/TBANO₃ in THF (Sample 11A) had on the surface of the Li-electrodes. The Li and TABF/TBANO₃ in THF (Sample 11A) treatment shows a significant increase in fluorine suggesting formation of lithium fluoride. A small increase in the concentration of nitrogen was assigned to mostly quaternary species and some neutral organic nitrogen. The total amount of carbon was reduced for Sample 11, including a small reduction in the amount of hydrocarbon and a significant reduction of carbonate species was observed from high-resolution spectra. Table 5 summarises the atomic % of various elements present on the surface of the as-supplied lithium metal (without precleaning) following immersion with fluorinating agent as shown in FIG. 17.

TABLE 5
Elemental quantification (Atomic %) for lithium electrode
(as supplied) immersed in THF (10), and lithium electrode (as supplied)
immersed TBAF/TBANO₃ (11) over 20 minutes.

| | | | | Li/F | Li/F comparison |
|---|---|---|---|---|---|
| # | Conditions | % Li | % F | ratio | (control/F agent) |
| 10A | Li (as-supplied) treated | 23.8 | 0.4 | 59.5 | — |
| | with THF | | | | |
| 11A | Li (as-supplied) treated | 37.1 | 35.0 | 1.1 | 54 |
| | with TBAF/TBANO₃ in | | | | |
| | THF | | | | |

As-supplied lithium metal immersed in THF (control; Sample 10A) comprised a surface with a significantly high Li/F ratio, highlighting the lack of fluorine enrichment on the surface. In contrast, the as supplied lithium metal immersed in TBAF/TBANO₃ in THF (Sample 11A) results in a ˜54-fold decrease of the Li/F ratio, which points to a significant increase in the amount of fluorine present in the ex-situ treated metal surface layer, as a result of the formation of the ex-situ SEI fluoride layer.

### Example 10: Use of Additives

The use of an additive, TBANO₃ in the THF/TBAF solution (TBANO₃ is THF soluble tetrabutylammonium nitrate) was then studied in a series of experiments using as-supplied lithium metal. As seen in FIG. 18, the TBAF/TBANO₃ in THF combination (Sample 6) is extremely effective in increasing the amount of lithium fluoride formed on the electrode surface using similar contact reaction times and temperature as the previous experiments. This is also evident by the high fluoride value compared to the data from the Li and THF (Sample 4) and Li and TBAF in THF (Sample 5) experiments. Table 6 summarises the atomic % of various elements present on the surface of the as-supplied lithium metal (without precleaning) following immersion with fluorinating agents (either with or without an additive) as shown in FIG. 18.

TABLE 6
Elemental quantification (Atomic %) for lithium surface (as
supplied) immersed in THF (4), Li and TBAF in THF (5), and Li and
TBAF/TBANO₃ in THF (Sample 6) over 20 minutes.

| | | | | Li/F | Li/F comparison |
|---|---|---|---|---|---|
| # | Conditions | % Li | % F | ratio | (control/F agent) |
| 4 | Li (as-supplied) in | 21.3 | 1.5 | 14.2 | — |
| | THF | | | | |
| 5 | Li (as-supplied) and | 16.3 | 10.1 | 1.6 | 8.9 |
| | TBAF in THF | | | | |
| 6 | Li (as-supplied) and | 32.7 | 23.2 | 1.4 | 10.1 |
| | TBAF/TBANO₃ in | | | | |
| | THF | | | | |

As-supplied lithium metal immersed in THF (control; Sample 14) comprised a surface with a high Li/F ratio. In contrast, the chemical treatment with TBAF in THF (Sample 5; fluorinating agent, no additive) or TBAF/TBANO₃ in THF (Sample 6; fluorinating agent with additive) results in ˜ 9 and ˜10-fold decrease of the Li/F ratio, respectively, pointing to an increase in the amount of fluorine present in the ex-situ treated metal surface layer. Additionally, the presence of the additive (TBANO₃) further increased the amount of fluorine present on the surface of the lithium (Sample 6).

### Example 11: Comparison with Non-Fluorinating Agents

The next set of experiments incorporate a comparison with a non-fluorinating agent. Perfluorohexane is a compound containing fluorine but one which is not a nucleophilic fluorinating agent, an electrophilic fluorinating agent, nor a radical fluorinating agent. Perfluorohexane is used as a representation of any compound that contains fluorine (such as PVDF, fluoropolymers and Teflon) to compare the reactivity of these stable fluoro-compounds with the fluorinating agents of the present disclosure.

All samples were treated outside glovebox. Li electrodes are used as-supplied without any surface cleaning. Li and THF only (i.e. solvent only—Sample 10A), Li and TBAF/TBANO₃ in THF (i.e. with fluorinating agent—Sample 11A) and perfluorohexane THF/Et₂O (48:2)) (i.e. with a compound containing fluorine but is a non-fluorinating agent—Sample 12A) were all used with the same metal to reagent mole ratio and 20 minute contact time to the chemical solutions. All samples one treated were subsequently cleaned and prepared for analysis as per previous experiments.

FIG. 17 compares the chemical treatments of Li and THF (Sample 10A), Li and TBAF/TBANO₃ in THF (Sample 11A) and Li and perfluorohexane in THF/Et₂O (48:2) (Sample 12A) on Li electrodes, and the atomic % of various elements present on the surface of the lithium metal are summarised in Table 7.

TABLE 7
Elemental quantification (Atomic %) for lithium electrode
(as supplied) immersed in THF (10), TBAF/TBANO₃ (11),
in THF and perfluorohexane (12) in THF over 20 minutes.

| | | | | Li/F | Li/F comparison |
|---|---|---|---|---|---|
| # | Conditions | % Li | % F | ratio | (control/F agent) |
| 10A | Li in THF | 23.8 | 0.4 | 59.5 | — |
| 11A | Li and TBAF/ | 37.1 | 35.0 | 1.1 | 54 |
| | TBANO₃ in THF | | | | |
| 12A | Li in | 24.1 | 1.5 | 16.1 | 3.7 |
| | perfluorohexane | | | | |
| | in THF/Et₂O | | | | |

Compared to the lithium metal (as supplied) in THF (control, Sample 10) the chemical treatment with TBAF/TBANO₃ in THF (Sample 11) results in ˜54-fold decrease of the Li/F ratio pointing to an increase in the amount of fluorine present in the ex-situ treated metal surface layer. In contrast the perfluorohexane immersion (Sample 12) only results in nominal change of the Li/F ratio.

To summarise, under room temperature and short contact times (20 minutes) comparing the Li and THF (Sample 10A) and the perfluorohexane in THF/Et₂O (48:2) (Sample 12A) treatments show little or no change of the fluorine, nitrogen, carbonate and hydrocarbon content. This suggests very little chemical reaction has occurred. In contrast, under similar conditions the Li and TABF/TBANO₃ in THF (Sample 11A) treatment shows a dramatic increase in fluorine and lithium content pointing to the formation of lithium fluoride (LiF). A small increase in the concentration of nitrogen was assigned to mostly quaternary species and some neutral organic nitrogen. The total amount of carbon was reduced for Sample 11, including a small reduction in the amount of hydrocarbon and a significant reduction of carbonate species was observed from high-resolution spectra.

Thus, the use of fluorine-containing compounds, which are not fluorinating agents (e.g. perfluorohexane), did not result in a SEI fluoride layer on the surface of the metal. Some compounds that contain fluorine cannot be considered “fluorinating” compounds in the context of the present disclosure. Typically compounds such as perfluorohexane have strong carbon-fluorine bonds which reduce their reactivity with lithium metal. The time frame and temperatures required to observe any “fluorination” onto the lithium metal is impractical and uneconomic. (see Basile, A. et al. Stabilizing lithium metal using ionic liquids for long-lived batteries. Nat. Commun. 7:11794, 2016). Neither of these extreme conditions (long contact times and higher temperatures lead to a controlled formation of a LiF SEI layer that is commercially viable or safe. The use of organic fluorinating compounds offers an approach to tune the composition of the SEI LiF layer by ex-situ solution based chemical processing.

### Example 12: Surface Treatment of Magnesium Electrodes

Magnesium metal was used instead of lithium in this example. The methodology was similar to the previous examples except for the following. Magnesium metal (used without precleaning) were treated with THF and TBAF/THF over 60 minutes outside of a glovebox. These experiments were also done side by side with the use of a non-fluorinating agent, see next experiment.

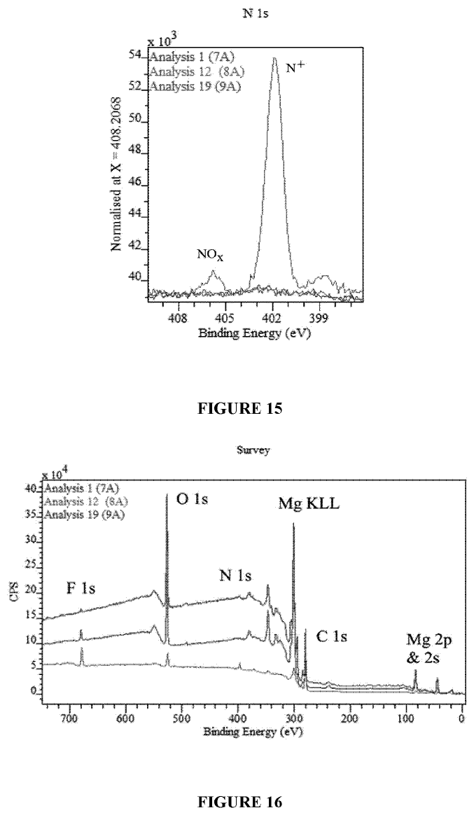

FIGS. 15 and 16 compares non-cleaned Mg metal in THF (i.e. solvent only—Sample 7A), TBAF and TBANO₃ in THF (i.e. with fluorinating agent—Sample 8A) and perfluorohexane in THF/Et₂O (48:2)) (i.e. with a compound containing fluorine but is a non-fluorinating agent—Sample 9A) under similar conditions over a 60 minute period, and the atomic % of various elements present on the surface of the lithium metal are summarised in Table 8.

TABLE 8
Elemental quantification (Atomic %) for magnesium metal surface immersed in THF
(7), TBAF/TBANO₃ in THF (8) and perfluorohexane (9) in THF over 60 minutes.

| | | | | | | | Mg/F | Mg/O |
|---|---|---|---|---|---|---|---|---|
| | | | | | | | comparison | comparison |
| | | | | | Mg/F | Mg/O | (F agent | (F agent |
| # | Conditions | % Mg | % F | % O | ratio | ratio | control) | control) |
| 7A | Mg in THF | 19.5 | 1.0 | 44.4 | 19.5 | 0.4 | — | — |
| 8A | Mg and | 9.0 | 4.7 | 8.1 | 1.9 | 4.7 | 10.3 | 25.8 |
| | TBAF/TBANO₃ | | | | | | | |
| | in THF | | | | | | | |
| 9A | Mg in | 22.3 | 3.5 | 44.4 | 6.4 | 0.5 | 3.0 | 1.2 |
| | perfluorohexane | | | | | | | |
| | in THF | | | | | | | |

Compared to the magnesium metal in THF (control; Sample 7A) the chemical treatment with TBAF/TBANO₃ in THF (Sample 8A) results in ˜10-fold decrease of the Mg/F ratio. While the perfluorohexane immersion (Sample 9A) also results in ˜4-fold decrease of the Mg/F ratio, the magnesium to oxygen (Mg/O) ratio essentially remains unchanged. In contrast, the Mg/O ratio is significantly increased in Mg immersed with TBAF/TBANO₃ (Sample 8A) by ˜26-fold. This indicates that in the case of the TBAF/TBANO₃ in THF treatment a generation of MgF₂ is observed however when treated with perfluorohexane, the oxide surface layer is still present and perfluorohexane is merely being adsorbed to the surface and not acting as a fluorinating agent, nor providing an ex-situ SEI fluoride layer.

In summary, for the two control conditions (e.g. solvent only (Sample 7A) or contact with a non-fluorinating agent, perfluorohexane (Samples 9A) the amount of observed total Mg and Mg as MgO are essentially the same suggesting even under this long contact time period little of the Mg has reacted with perfluorohexane. In contrast, considerably less Mg as MgO surface is present when treated with TBAF/TBANO₃ in THF (Samples 8A) suggesting MgO has been removed from the surface. In addition, there is still a significant amount of carbonate present in the samples of Mg in THF and Mg in perfluorohexane in THF, while no carbonate is observed in the Mg sample which has been subjected to TBAF/TBANO₃ in THF.

Regarding the fluorine concentration, FIG. 16 shows that while the initial Mg sample does contain a relatively minor amount of fluorine, the fluorine concentration increases significantly when subjected to the TBAF/TBANO₃ in THF. FIG. 15 also confirms the presence of nitrogen species for TBAF/TBANO₃ in THF treatment. Closer examination of the different types of nitrogen species by XPS as shown in the FIG. 15 shows different types on nitrogen present, which is ascribed to a small amount of NOx and neutral organic nitrogen species, and majority quaternary amine species, which are not present in the Mg in THF sample.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, methods, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

The present application claims priority from AU 2020900286 filed 3 Feb. 2020, the entire contents of which are incorporated herein by reference.

1. A method for forming an ex-situ SEI fluoride layer on the surface of a metal or an electrode thereof, the method comprising the step of contacting the surface of the metal with an organic solvent preparation comprising one or more fluorinating agents.

2. (canceled)

3. The method of claim 1, wherein the metal is a metal electrode.

4.-8. (canceled)

9. The method of claim 1, wherein the metal is an alkali metal or an alkali earth metal.

10. The method of claim 1, wherein the metal is selected from the group consisting of lithium, magnesium, calcium, sodium, aluminium, and potassium metal.

11. (canceled)

12. The method of claim 1, wherein the metal is lithium metal.

13. (canceled)

14. The method of claim 1, wherein the fluorinating agent is selected from the group consisting of a nucleophilic fluorinating agent, an electrophilic fluorinating agent, a radical fluorinating agent, and any combinations thereof.

15. The method of claim 1, wherein the fluorinating agent is selected from an NF fluorinating agent containing an ionic NF bond or covalent NF bond.

16. The method of claim 1, wherein the fluorinating agent is a compound of Formula 1a: 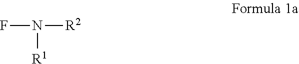
wherein
R₁ and R₂ are each independently selected from an optionally substituted alkyl or an electron withdrawing group, or R₁ and R₂ together form an optionally substituted heterocyclic ring.

17. The method of claim 16, wherein the fluorinating agent is a compound of Formula 1a selected from the group consisting of an N-fluoroarylsulfonimide such as N-fluorobenzenesulfonimide (PhSO₂)₂NF; NFSI), N-fluoroalkylsulfonamides, N-fluoro-o-benzenesulfonimide (Ph(SO₂)₂NF; NFOBS), N-fluorosultams, and N-fluorooxathiazinone dioxide.

18. (canceled)

19. The method of claim 1, wherein the fluorinating agent is an ionic salt compound of Formula 1b: 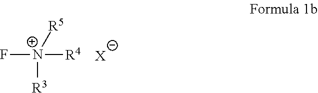
wherein
R₃, R₄ and R₅ are each independently selected from an optionally substituted alkyl or together form an optionally substituted monocyclic or bicyclic heterocyclic ring; and
X⁻ is a counter anion selected from a triflate, borate, and phosphate, each of which may contain one or more fluoro atoms.

20. The method of claim 19, wherein the fluorinating agent is an ionic salt compound of Formula 1b selected from N-fluoropyridinium triflate, N-fluoro-2,4,6-trimethylpyridinium triflate, N-fluoro-2,4,6-trimethylpyridinium tetrafluoroborate, N-fluoro-2,6-dichloropyridinium tetrafluoroborate, N-fluoro-2,6 dichloropyridinium triflate, N-fluoropyridinium pyridine heptafluorodiborate, N-fluoropyridinium tetrafluoroborate, N-chloromethyl-N′-fluorotriethylenediammonium bis(tetrafluoroborate) (Selectfluor®), N-chloromethyl-N′-fluorotriethylenediammonium bis(hexafluorophosphate), and N-chloromethyl-N′-fluorotriethylenediammonium bis(triflate).

21. (canceled)

22. The method of claim 1, wherein the fluorinating agent is an ionic compound of Formula 2a:
R₆R₇R₈N⁺F⁻ Formula 2a
wherein
R₆, R₇ and R₈ are each independently selected from an optionally substituted alkyl or together form an optionally substituted monocyclic or bicyclic heterocyclic ring.

23. The method of claim 22, wherein the fluorinating agent is tetrabutylammonium fluoride (TBAF).

24. The method of claim 1, wherein the organic solvent preparation comprises one or more aprotic organic solvents.

25. (canceled)

26. The method of claim 1, wherein the organic solvent preparation comprises one or more aprotic solvents selected from any one or more of 1,2-dimethoxyethane, diglyme, triglyme, tetraglyme, ethylene carbonate, propylene carbonate, dimethyl carbonate, tetrahydrofuran, and dioxolane.

27. (canceled)

28. (canceled)

29. The method of claim 1, wherein the step of contacting the surface of the metal with an organic solvent preparation is provided at a temperature between about −100° C. to 150° C. or between a temperature between about 0° C. to 50° C.

30. (canceled)

31. The method of claim 1, wherein the surface of the metal or electrode thereof is cleaned prior to contacting the surface of the metal with the organic solvent preparation comprising one or more fluorinating agents.

32. An ex-situ SEI fluoride layered metal or an electrode thereof prepared by the method according to claim 1.

33. A method of assembling an electrochemical cell comprising a metal electrode, whereby the steps comprise:
treating a metal or an electrode thereof according to claim 1 to form an ex-situ SEI fluoride layered metal or electrode thereof;
optionally preparing an ex-situ SEI fluoride layered metal electrode from the ex-situ SEI fluoride layered metal; and
assembling the ex-situ SEI fluoride layered metal electrode into an electrochemical cell.

34. (canceled)

35. An electrochemical cell comprising:
a negative electrode comprising or consisting of an ex-situ SEI fluoride layered metal or electrode thereof according to claim 1;
a positive electrode comprising a positive electrode active material; and
an electrolyte comprising one or more electrolyte solvents.
